(19) United States
(12) Patent Application Publication
(10) Pub. No.: US 2007/0178561 A1
(43) Pub. Date: Aug. 2, 2007

(54) Anti-hiv-1 compounds based upon a conserved amino acid sequence shared by gp160 and the human cd4 protein

(76) Inventors: Porter W. Anderson, Key Largo, FL (US); J. Ellis Bell, Richmond, VA (US)

Correspondence Address: NEEDLE & ROSENBERG, P.C., SUITE 1000, 999 PEACHTREE STREET, ATLANTA, GA, 30309-3915, US

(21) Appl. No.: 10/555,810

(22) PCT Filed: May 10, 2004
(86) PCT No.: PCT/US04/14650

§ 371 Date: Nov. 20, 2006

Related U.S. Application Data
(60) Provisional application No. 60/468,847, filed on May 8, 2003.

(51) Int. Cl. C12Q 1/68; C12P 19/34

(52) U.S. Cl. 435/91.1; 435/6

(57) ABSTRACT

Disclosed are compositions and methods that relate generally to human immunodeficiency virus (HIV), and more particularly to the agents and their identification and use of anti-HIV agents which interfere with binding of a target amino acid sequence within glycoprotein 160 of HIV-1 to its ligand. Further disclosed is a composition comprising the molecule and a suitable carrier, and a method of decreasing interaction of human immunodeficiency virus with a host cell, the method comprising exposing one or both of the virus and the host cell to the molecule.

### I. ACKNOWLEDGEMENTS

This application claims the benefit of U.S. Provisional Application No. 60/468,847, filed May 8, 2003. This application is herein incorporated by reference in its entirety.

### II. BACKGROUND

Human Immunodeficiency Virus (HIV) exists in at least two major forms, HIV-1 and HIV-2. HIV-1 is thought to be more virulent than HIV-2 in humans and is the major agent of Acquired Immunodeficiency Syndrome (AIDS), a major public health problem. HIV-2, although eventually fatal in many cases, has a slower progression. Simian Immunodeficiency Viruses (SIV) are found in various non-human primates and genetically resemble HIV-2; however, SIV-CZ, from chimpanzees, is believed to be very closely related to HIV-1 and MIVs (mammalian immunodeficiency viruses) are found in many mammals, such as feline.

The complex replication cycle of HIV has been characterized in its overall outline. The virus contains at least twelve genes, and the roles of protein or nucleic acid products of the genes are generally known. One gene known to be important in HIV virulence is env. Its product, called glycoprotein (gp) 160, is externally situated and is part of the viral “envelope” or membrane. gp160 is a precursor that is proteolyzed into two discrete products that remain functionally connected; gp120, which specifies the binding to the CD4 receptor protein and the essential co-receptors such as CCR5 or CXCR4 (originally called fusins), and gp41, which controls the subsequent fusion of viral and cellular membranes. gp41 contains two sequences referred to as transmembrane (TM) domains that are able to insert into host cell or viral membranes. The TM domain nearer the amino terminus is called the fusion domain, since extensive study has shown it to be critical for the fusion process. Fusion occurs when a virus particle enters the host cell and when a virus-infected cell (expressing gp 160 at its surface) fuses with uninfected, susceptible cells in a process called syncytium formation. The processes in which newly formed virus nucleocapsids attach to the interior of the cell membrane, become enveloped, and bud off as free virus particles may also partake of the fusion process.

The function of the second TM domain of gp41, amino acid residues approximately 676-706 (this region varies in number according to the HIV 1/2 type but is always present), has been less studied, but also appears to have a role in membrane fusion as well as insertion. (Note that the numbering of residues refers to the intact gp160; numeration in various publications varies slightly; the numeration of Helseth et al, Journal of Virology 64:6314, 1990 is used herein unless otherwise noted.) An arginine residue at 696 was noted to be highly conserved and the only known variation is a lysine which is also positively charged. (Owens et al, Journal of Virology 68:570, 1994).

Mutational replacement of this (positively charged) arginine with the non-charged amino acid serine somewhat diminished capacity for replication and fusion measured as syncytium formation, and replacement with a four-amino-acid insert strongly diminished these activities (Helseth et al, above). Amino acid substitutions at 687-689 and at 697-699 likewise strongly inhibited replication and syncytium formation (Gabuzda et al, Journal of Acquired Immune Deficiency Syndromes 4:34, 1991). Replacement of arginine 696 with the highly hydrophobic amino acid leucine or truncation eliminating amino acids carboxy terminal from arginine 696 strongly diminished syncytium formation without interfering with the capacity of the modified proteins to associate with the host cell membrane; truncation of amino acids carboxy terminal from 692 or from 683 eliminated the latter capacity as well (Owens et al, above). Thus the second TM domain—the object of our study described below—was known to be functionally important for HIV, but the structural basis was not understood. The CD4 receptor and the co-receptors called fusins, in addition to the extracellular domains recognized by gp120, have TM domains anchoring them in the cell membrane.

Disclosed are compositions and methods that bind a notch sequence or mimic a notch sequence as disclosed herein, and which can inhibit function of the gp160 (gp120) HIV molecule.

### III. SUMMARY

Disclosed are compositions and methods that relate generally to human immunodeficiency virus (HIV), and more particularly to the agents and their identification and use of anti-HIV agents which can interfere with binding of a target amino acid sequence within glycoprotein 160 of HIV-1 to its ligand.

For example, disclosed are molecules capable of interfering with binding of a target amino acid sequence within the second TM region of gp41 of HIV-1 to its ligand, wherein the target is an amino acid sequence selected from the group consisting of SEQ ID NO:13, SEQ ID NO:14, and SEQ ID NO:15, where X is any amino acid that allows the sequence to form a helix and be embedded in a membrane environment, and these sequences represent variations of a structurally similar consensus sequence in gp41 of HIV-1 which form a glycine-surfaced discontinuity or “notch” in the alpha helix. Such molecules include those which interfere by binding to the target, those which interfere by binding to its ligand (these molecules mimic the target), and those which interfere by binding to viral nucleic acid encoding the target, and prevent synthesis of the target.

Disclosed are compositions comprising the molecule of the subject invention and a suitable carrier, as well as a method of decreasing interaction of human immunodeficiency virus with a host cell, the method comprising exposing one or both of the virus and the host cell to a disclosed molecule.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a computer-generated model of portions of the second transmembrane region of HIV-1 gp41.

FIG. 2 shows a computer-generated model of portions of the second transmembrane region of HIV-2 gp41.

FIG. 3 shows a computer-generated model of portions of the second transmembrane region of the corresponding region of human CD4.

FIG. 4 shows binding together or “docking” of the above-described transmembrane regions of HIV-1 and CD4.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Although embodiments have been depicted and described in detail herein, various modifications, additions, substitutions and the like can be made.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular notch structural motif is disclosed and discussed and a number of modifications that can be made to a number of molecules including the notch structural motif are discussed, specifically contemplated is each and every combination and permutation of notch structural motif and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

B. Compositions

Disclosed are compositions comprising suitable carriers, as well as a method of decreasing interaction of human immunodeficiency virus with a host cell. The methods comprise exposing one or both of the virus and the host cell to the molecule. Descriptions and means of identifying and/or screening for such a molecule can be performed. It is also understood that there is a variety of structural information provided herein, including atomic coordinates, and that this information can be used to define the disclosed compositions, including the notch binders, HIV infectivity inhibitors, and inhibitors of the CD4-gp160 interaction. Disclosed are compositions that interfere with HIV infectivity, by for example, interfering with gp160 function, through for example, preventing gp160 coordination of cell entry by HIV.

1. Target or Viral Notch Sequence

Disclosed herein the Human Immunodeficiency Virus, Type 1 (HIV-1) contains a structurally highly conserved amino acid sequence in the second transmembrane segment of the envelope glycoprotein (gp 160). This highly conserved amino acid sequence structurally resembles a sequence present in both the transmembrane segment of the virus receptor protein of susceptible host cells (CD4 protein in the case of HIV-1) and with respect to the conserved glycines, the co-receptors termed fusins (chemokine receptor family). The sequence in the case of HIV-1 gp 160 is SEQ ID NO:1: IVGGLVGL, and corresponds to residues numbered 688-697. (This can also be understood as 683-690 in the full sequence of gp 160 published by Ratner et al. It is understood that differing numbering conventions can be used to define this region, depending on what portions of the gp160 protein are present, but that the sequences represented by this region can readily be understood as disclosed herein.) The sequence in the case of HIV-1 gp 160 can also be extended to SEQ ID NO:35: FMIVGGLVGLRIV, and corresponds to residues numbered 686-699. (This can also be understood as 681-692 in the full sequence of gp 160 published by Ratner et al.). Disclosed herein this sequence or its structural equivalent is present in all 690 of the HIV-1 isolates examined and the structurally similar sequence SEQ ID NO:2: VLGGVAGL is present in human and other primate CD4 proteins and that the structurally similar sequence SEQ ID NO:3: IGYFGGIF is present in the co-receptor family known as the fusins; and that the structurally similar sequence SEQ ID NO:4: CVGGLLGN is present in the protein, OPRY-HUMAN, present in the brain. (CD4, Maddon, P. J., et al., Cell 42 (1), 93-104 (1985); fusins, Charo, I. F., et al., Proc. Natl. Acad. Sci. U.S.A. 91 (7), 2752-2756 (1994); OPRY, Wick, M. J., et al., Brain Res. Mol. Brain Res. 32 (2), 342-347 (1995), all of which are herein incorporated at least for material related to the denoted proteins, including sequence and structure information.) Also disclosed herein the sequence in SEQ ID NO:1 and 35 or its structural equivalent is present in all 690 of the HIV-1 isolates examined and the structurally similar sequence SEQ ID NO:36: ALVLGGVAGLLLF is present in human and other primate CD4 proteins

These octapeptide and triskadecapeptide sequences lie within a transmembrane (lipid bilayer-inserting) region of each protein and can form a glycine-surfaced discontinuity or “notch” in the chain typically if the peptide, as shown herein, is in alpha helical configuration. This is consistent with the viral notch being crucial in membrane insertion and fusion, and thus forming a critical binding site in the replication cycle of HIV-1. The site thus provides a target for classes of antiviral agents. Data disclosed herein are consistent with the notch region of the virus interacting with the notch region of the receptor proteins during replication or the notch regions of the various proteins having a common ligand.

2. Compositions that Bind the Notch

The HIV-1 notch is a functional site. The notch region is a site for targeting therapeutic reagents, i.e., a molecule interfering with the viral notch could be used to inhibit HIV-1 replication.

Disclosed are notch inhibitors that in certain embodiments can be anything that competes with a notch-notch interaction, or binds a notch region. For example, the inhibitors could be a peptide, antibody, protein, small molecule, or functional nucleic acid. Disclosed are molecules that can interfere with the viral life cycle.

Physically the notch in certain embodiments can be described as 4-5A deep, 12-13A wide with a depth of 8-9A. For example, the notch sequence in certain embodiments can be described as XXXXGGXXGXYXX- where X is any hydrophobic residue and Y is R or any hydrophobic residue. This 13mer defines the three dimensional structure of the notch as found in CD4 or HIV1. Physically the notch can be described as a hydrophobically lined cavity with a length (defined from N to C terminal atoms- of 10-14A, a width of ˜9.5A, with a 5A central groove lined by atoms capable of hydrogen bond or dipolar interactions, and a depth of 4-6A) This is defined in space by the three dimensional coordinates for the second TM helix of gp41 as discussed in Tables 3 and 4.

The Notch inhibitors can bind with Kds of 10⁻M, 10⁻⁴ M, 10⁻⁵ M, 10⁻⁶ M, 10⁻⁷ M, 10⁻⁸ M, 10⁻⁹ M, or 10⁻¹⁰ M, or 10⁻¹¹ M.

The molecules can be any sized molecule that is capable of binding to the above described “notch” and inhibiting its biological activity, or binding to the putative interacting partner of the target and preventing interaction with the target and thus acting as a notch inhibitor as described herein. The disclosed peptides can be computationally docked, as disclosed herein, with the target and can be notch inhibitors if they could be delivered to the site of action effectively. For example, the disclosed peptides that function as notch inhibitors can be any length. The disclosed peptides can be greater than or equal to 8, 9, 10, 11, 12, 13, 14, 15, 16, 17, 18, 19, 20, 21, 22, 23, 24, 25, 26, 27, 28, 29 30, 35, 40, 45, 50, 60, 70, 80 90, or 100 amino acids long. The peptides that are notch inhibitors can also be peptides of any length, but can be between about 10 to about 50 amino acids in length. The peptides can be less than or equal to about 200 amino acids, 150 amino acids, 125 amino acids, 100 amino acids, 75 amino acids, 50 amino acids, 40 amino acids, 30 amino acids, 25 amino acids, 20 amino acids, 15 amino acids, or 10 amino acids. Where the peptide is functioning to form a notch structure, what is required is that the peptide be able to form an alpha helix that forms the notch structure as discussed herein. It is also preferred that the notch structure comprise a sequence capable of inserting into a membrane region.

The disclosed molecules can be identified in numerous ways. For example, the information disclosed herein that the binding the notch and interfering with the notch function is desirable can be utilized to identify molecules that inhibit HIV infectivity.

It is also understood that modifications can be made to the disclosed molecules that can increase the affinity of the molecule for the notch region. For example, negatively charged residues can be added to the disclosed molecules such that the negatively charged residues interact with the positively charged arginine residue next to the notch. Another means for increasing the affinity of notch inhibitors is by adding covalent links at intervals of i to i+7 to stabilize the alpha-helical conformation (Judice et al, Proc Nat Acad. Sci 94:13426, 1997).] Still another is addition of a peptide “leader” or entry sequence to facilitate membrane penetration. A number of different such peptides are known. For example, peptides such as poly arginine can be used.

The disclosed compositions can also be modified to improve solubility in biological membranes, such as by capping terminal amino acids to suppress charge. Also disclosed are small molecules, such as “peptoid” compounds (Simon et al, Proceedings of the National Academy of Science, USA 89: 9367, 1992, herein incorporated by reference at least for material related to peptoids molecules and their use and structure).

Disclosed are notch inhibitors designed to reduce degradation, such as proteolytic degradation by the host For example, D amino acids can be substituted for L amino acids to increases resistance to proteolytic degradation. Also disclosed are notch inhibitors that have the same sequences of side chains but which are synthesized containing retro-inversion peptide bonds which also exhibit similar antiviral activity but have improved stability to proteolytic degradation.

The disclosed molecules can be combined with structural refinements that can increase specificity, affinity, membrane solubility, or biological efficacy (stability and bioavailability).

a) Peptides

Disclosed are peptides that are able to bind a notch sequence. These peptides can be notch sequences, sequences that mimic a notch sequence, or sequences that are able to make the appropriate contacts with the notch sequence structural configuration so that binding between the peptide and the notch sequence occurs.

Disclosed are molecules capable of interfering with binding of a target within HIV-1 gp160 to its normal ligand, wherein the target is an amino acid sequence selected from the group consisting of 13-15 or a structurally related sequence. In a further embodiment, the target is an amino acid sequence selected from the group consisting of SEQ ID NO:22, SEQ ID NO:23, and SEQ ID NO:24, or a structurally related sequence. In another embodiment, the target is an amino acid sequence selected from the group consisting of SEQ ID NO:16, SEQ ID NO: 17, and SEQ ID NO:18, or a structurally related sequence. In a still further embodiment, the target is an amino acid sequence selected from the group consisting of SEQ ID NO:19, SEQ ID NO:20, and SEQ ID NO:2 1, or a structurally related sequence.

These sequences represent a highly conserved (consensus) sequence within the second transmembrane segment of the envelope glycoprotein gp160 (gp41 portion) that has been identified in accordance with the subject invention. This consensus sequence of the glycine motif or its structural equivalent was found in all 690 of the HIV-1 isolates examined, but was not found in any of 29 examined HIV-2 isolates (which are less virulent in humans). The sequences, or, indirectly, the host cell ligand with which they interact, or the nucleic acid encoding the amino acid sequences, thus represent a target for anti-HIV-1 molecules, these anti-HIV-1 molecules being useful in the treatment and/or prevention of diseases and/or disorders associated with HIV-1 (including Acquired Immunodeficiency Syndrome; AIDS).

Disclosed are molecules that bind to the viral notch sequence or bind to ligands that normally bind the notch and therefore, prevent the notch-ligand interaction. For example, peptides comprising a notch sequence (or its “mirror” sequence) are disclosed. These types of molecules are capable of inhibiting a notch-notch interaction or a notch interaction to another type of protein through, for example, competitive inhibition. Molecules containing a notch sequence or its mirror are shown herein to be able to dock with the HIV-1 notch sequence. This is consistent with these molecules when having access to the notch sequence being able to interact with the notch sequence and act as competitive inhibitors of other sequences that interact could interact with the notch sequence. Any peptide comprising a notch sequence can be used to interact with a notch sequence. For example, the peptide EGGIVGGVAGLLL (SEQ ID NO 7) and EGGIVGGVAGLLL[G]ₓ[R]_{y} (SEQ ID NO 34), represents an extended version of a notch octapeptide. The dipeptide LL added at the carboxyl terminus is intended to stabilize a helical structure and is present also in CD4. [G]ₓ is a flexible glycyl linker. [R]_{y} is a series of arginines to facilitate binding to the negatively charged surface of phospholipid membranes. At the amino terminal is added EGG, a flexible diglycyl linker plus glutamate (E), a negatively charged amino acid that will increase affinity by charge-charge bonding to the position 9 arginine in HIV-1. The alpha amino terminus of the peptide is blocked by acylation to remove the formal charge and thus increase membrane solubility

Also disclosed are peptides comprising Z(X)n)IVGGVAGLLL (SEQ ID NO 25) or Z(X)n)IVGGVAGLLL[G]_{X}[R]_{Y}, (SEQ ID NO:34) which are extended versions of a notch octapeptide. At the amino terminal is added Z(X)n, where (X)n is a flexible linker and Z is a moiety capable of optimizing interaction with the completely conserved positively charged amino acid (R/K) in the target, for example glutamate (E), a negatively charged amino acid that will increase affinity by charge-charge bonding to the R/K at position 9 of SEQ ID NO:6. Disclosed herein, a numbering system is where 1 is at the amino terminus of the octapeptide sequence, making arginine in HIV-1 position 9. The alpha amino and carboxyl termini of the peptide can be blocked by acylation and amidation respectively.

Also disclosed are peptides comprising QPMALIVGGVAGLLLFIGLGIFFCVR (SEQ ID NO: 8), which represents an extended version of SEQ ID NO:7. The termini, however, are unblocked and thus charged, so as to span and anchor the peptide in the cell membrane. These peptides can bind a notch structure based on molecular modeling studies.

Also disclosed are peptides that are the mirror sequence of the notch sequence. For example, SEQ ID NOs: 13-15 and 22-25, and SEQ ID NO:7 have the -G-G-X-X-G- motif and can be reversed to -G-X-X-G-G-. This motif, present in the protein fusin, likewise would contain the notch structure.

Peptides that form a notch type sequence, which are not themselves the consensus notch sequence are disclosed. In certain embodiments the notch is defined by the glycines and there position relative to each other, if they are in a stable structure, the notch structure is predicated by the glycine sequences, the dimensions of notch are based on what are before and after the glycines. These sequences are capable of forming a helix, and typically would not for example, include a proline. In certain embodiments any sequence of 5 or more amino acids that contains G-X-X-G-G or G-G-X-X-G and is capable of forming a helix are disclosed. The notch can be defined by the adjacent residues. If you want a generic description of a sequence with a notch use X-G-X-X-G-G-X or X-G-G-X-X-G-X where X is any amino acid other than Glycine Alanines can be contained, for example, in the first or last G of either sequence, within the molecules. These molecules are capable of forming the appropriate three dimensional notch structure and could bind the notch sequence. For example, disclosed is IVGGLVGL (SEQ ID NO 1), the HIV-1 notch octapeptide. In SEQ ID NO:1 the amino- and carboxyl termini can be acyl- and amnide-blocked respectively and thus not charged.

Also disclosed are peptides comprising MIVGGLVGLR (SEQ ID NO:9), a peptide consisting of the HIV-1 octapeptide with its contiguous amino-terminal methionine (M), which can bind the notch structure, and its contiguous arginine (R). The amino- and carboxyl termini can be blocked and thus not charged. Residues having a charge, for example a D sidechain, such as the arginine in SEQ ID NO:9 can increase the solubility of the molecule in a carrier, such as a pharmaceutically acceptable carrier.

Also disclosed are peptides comprising YIKIFMIVGGLVGLRIVFAVLSIVNR (SEQ ID NO:10), which represents a longer extended version of the gp160 notch peptide.]

The peptides disclosed herein can be synthesized. The termini of the disclosed peptides can be blocked or unblocked. Typically, when the termini are blocked the peptide will be uncharged relative to the termini of the peptide. For example, the carboxy termini can be blocked through an acylation reaction and the amino termini can be blocked through an amidation reaction. When the termini are unblocked this can aid in spanning the membrane, through charge interactions which can anchor the peptide in the membrane.

Interference with the replication cycle by oligopeptides that mimic sites on viral or cell receptor proteins have been examined for HIV but these peptides are not alpha helical and do not have activity with the notch as disclosed herein. (U.S. Pat. No. 5,444,044 with molecule SJ2176 of Jiang, which are coil of coils, and are not functional molecules as disclosed herein and Wild et al., AIDS Research & Human Retroviruses 11:323, 1995 where DP178=T20 of Trimeris, neither interact with the notch but interferes with a conformation change in soluble gp160).

It is understood that in certain embodiments, molecules comprising 676-702 plus KKKC are not notch inhibitors. Jiang et al. (Nature, 365:113, 1993) tested a peptide described as “683-707KKKC” and found it bound gp160 but it does not inhibit viral growth in vitro viral cell growth assays as disclosed herein using p24. It is likely that the kkkc, since it is positively charged, lowers entrance into a bilayer environment, however, as disclosed herein, the notch may need to be in the bilayer environment to function as a anti-viral. Therefore, non-charged, hydrophobic molecules are preferred, at least for the portion of the molecule which will be thought to be in the membrane. Arginine appears to be critical as it is highly conserved, and likely anchors the helix in the membrane and can interact with negative charges in the phospholipid.

Furthermore, by the Helseth et al. numeration this corresponds to gp160 residues 676-702 plus a (non-natural) linker extension containing three lysine residues (K) and a cysteine residue (C). Computer modeling of this peptide consisting of amino acids 676-702 plus KKKC (SEQ ID NO:29, TNWLWYIKLFIMIVGGLVGLRIVFAKKKC) showed that this peptide does not form a stable alpha helix and hence stable notch structure. This peptide does not have activity as a notch inhibitor, as disclosed herein. The three lysines (K) and cysteine (C) destabilize the helix, resulting in less notch present on the peptide to interact with another notch region.

b) Antibodies

Also disclosed are antibodies or related molecules able to bind to the notch region and act as notch inhibitors. It is understood that in certain embodiments the antibodies areor contain hydrophobic regions on them. Disclosed are antibodies able to bind to the target sequence (such as a polyclonal or monoclonal antibody, including chimeric or humanized antibodies). Suitable molecules capable of binding to the target can be identified by any means. For example, a peptide can be synthesized which includes the target amino acid residues, such as a sequence representing the notch. The chemically synthesized peptide can be conjugated to bovine serum albumin and used for raising polyclonal antibodies in rabbits. Standard procedures can be used to immunize the rabbits and to collect serum; as described herein. Polyclonal antibody can be tested for its ability to bind to gp160 (or the peptide fragment). For polyclonal antibody that shows a high affinity binding to gp160, functional studies can then be undertaken for reduction in gp160. Fragments (such as Fab, Pc, F(ab′)₂) of the polyclonal antibody can be made if steric hindrance appears to be preventing an accurate evaluation of more specific modulating effects of the antibody. For example, the antibodies can bind the notch structural motif.

Alternatively, monoclonal antibody production can be carried out using BALB/c mice. Immunization of B-cell donor mice can involve immunizing them with antigens mixed in TiterMax™ adjuvant as follows: 50 micrograms antigen/20 microliters emulsion×2 injections given by an intramuscular injection in each hind flank on day 1. Blood samples can be drawn by tail bleeds on days 28 and 56 to check the titers by ELISA assay. At peak titer (usually day 56) the mice can be subjected to euthanasia by CO₂ inhalation, after which splenectomies can be performed and spleen cells harvested for the preparation of hybridomas by standard methods.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain notch binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boemer et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Disclosed are hybridoma cells that produces the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises one or more of SEQ ID NOs:1-25. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of a gp160, such as the notch structural motif, expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of notch antibody as fusion proteins. The antigen is produced by inserting a gene fragment in-frame between the signal sequence and the mature protein domain of the notch antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against, for example the notch structural motif. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for a notch structural motif and another antigen-combining site having specificity for a different antigen of, for example, gp160.

In vitro methods are also suitable for preparing monovalent antibodies. Digestion of antibodies to produce fragments thereof, particularly, Fab fragments, can be accomplished using routine techniques known in the art For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction Lawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with a notch structural motif. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragment thereof and one or more reagents for detecting binding of the antibody or fragment thereof to the notch structural motif. The reagents can include, for example, fluorescent tags, enzymatic tags, or other tags. The reagents can also include secondary or tertiary antibodies or reagents for enzymatic reactions, wherein the enzymatic reactions produce a product that can be visualized.

c) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of a notch structural motif or the genomic DNA of a notch structural motif or they can interact with the polypeptide of a notch structural motif. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

It is understood that in certain embodiments functional nucleic acids that specifically target the mRNA encoding the notch are preferred because the notch is a highly conserved protein motif. The highly conserved protein motif has a defined set of mRNAs or RNA or DNA that can code for the protein motif. Thus, this region represents a preferred target for mRNA or viral genome destruction because the viral genome or mRNA should be more conserved than in other areas of the genome, in which the protein sequence can vary which allows for even greater variation at the nucleic acid level encoding that protein. For example, degenerate target molecules, such as antisense, ribozymes, and RNAi can be used and would have the advantage of targeting a region that was more resistant to variation. A rapidly evolving virus typically needs to conserve highly conserved protein structural features, which limits the variation that can take place at the genomic level.

It is also understood that the disclosed nucleic acids can be used for RNAi or RNA interference. It is thought that RNAi involves a two-step mechanism for RNA interference (RNAi): an initiation step and an effector step. For example, in the first step, input double-stranded (ds) RNA (siRNA) is processed into small fragments, such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. Disclosed are RNA hairpins that can act in RNAi.

RNAi has been shown to work in a number of cells, including mammalian cells. For work in mammalian cells it is preferred that the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells. For description of making and using RNAi molecules see See, e.g., Hammond et al., Nature Rev Gen 2: 110-119 (2001); Sharp, Genes Dev 15: 485-490 (2001), Waterhouse et al., Proc. Natl. Acad. Sci. USA 95(23): 13959-13964 (1998) all of which are incorporated herein by reference in their entireties and at least form material related to delivery and making of RNAi molecules.

For the highly conserved heptapeptide sequence V/I-G-G-L/I-V/I-G-L/I a degenerate set of RNAi molecules would consist of sequences shown in Table 9.

TABLE 9

| 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|
| C | A | A | C | C | A | C | C | A | C | A | A | C | T | A | C | C | A | G | T | A |
| T | | T | | | T | | | T | T | | T | | C | T | | | T | T | | T |
| | | C | | | C | | | C | | | C | | | C | | | C | | | C |
| | | G | | | G | | | G | | | G | | | G | | | G | | | G |

Where at each position the indicated variation is allowed. Because of the mechanism of synthesis of degenerate oligonucleotides this set is as easily synthesized as any 21 mer. It is understood that RNAi molecules can be delivered and used as understood in the art, including delivery via vectors and with expression from Pol III promoters. It is understood that the sequences in Table 8 can be made from RNA, can be made as double stranded RNA, can be made as DNA or double stranded DNA, as well as chemically synthesized variants of all of these. In certain embodiments, siRNAs can be made as short hairpins, and that these short hairpins could be added to the sequences in Table 8, by adding a loop region, along with the sequence and complementary sequence. For example, a loop region could be 5′-TTTTTTTTT-3′, 5′-TATATATATA-3′, 5′-TCTCTCT-3′, or any combination of these, up to for, example, a 20 mer loop. It is also understood that all molecules in Table 8 can be made as any stem loop or double stranded molecule, including any 3′ or 5′ overhang as discussed herein. RNAi molecules can be delivered as double stranded RNA, single stranded RNA, made either enzymatically as well as chemically, and they can also be produced via vectors expressing them. It is understood that if the sequences in Table 8 are RNA, T will become U.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS (dimethylsulfoxide) and DEPC (diethylpyrocarbonate). It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437. It is understood that antisense molecules having the sequences disclosed in Table 9 are also disclosed, but that these can be optimized as deoxyribonucleotide molecules as well as RNA molecules or modified forms of these.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of notch aptamers, the background protein could be serum albumin. Representative examples of how to make and use aptamers to bind a variety of different target molecules can be found in the following non-limiting list of U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acids. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos.: 5,631,115, 5,646,031, 5,683,902, 5,712,384, 5,856,188, 5,866,701, 5,869,339, and 6,022,962), and tetrahymena nbozymes (for example, but not limited to the following U.S. Pat. Nos.: 5,595,873 and 5,652,107). There are also a number of ribozymes that are not found in natural systems, but which have been engineered to catalyze specific reactions de novo (for example, but not limited to the following U.S. Pat. No.: 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes nbozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos.: 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos.: 5,168,053, 5,624,824, 5,683,873, 5,728,521, 5,869,248, and 5,877,162.

d) Compositions Identified By Screening with Disclosed Compositions Combinatorial Chemistry and Methods of Identifying

The information disclosed herein provides targets for therapeutic molecules. These therapeutic molecules can be identified using any method, including for example, combinatorial chemistry techniques, as well as molecular modeling. One aspect of the methods of identification is that certain sequences in gp160 are found to be highly conserved and that these sequences form a unique structure which is associated with HIV infectivity. Various methods that utilize this information can be employed. For example, since the three dimensional structure of this conserved notch region is known the structure can be used for modeling coordinates within which candidate binding molecules can be docked. The identification methods can be used with any molecule, depending on the disclosed methods. It is understood that molecules which inhibit the viral replication through interacting with the viral nucleic acid, through for example, antisense or nbozymes technology, can also be identified which specifically interact at the nucleic acid encoding the notch region of the polypeptide, and are disclosed.

For example, small molecule notch inhibitors can be identified as discussed herein using, for example, combinatorial chemistry and libraries of molecules to identify those that bind the notch region. For example, “peptoids” compounds (Simon et al, Proceedings of the National Academy of Science, USA 89: 9367, 1992) can be used for screening. Screening methods can include, for example, attaching the notch region to a support, such as a 96 well plate, and isolating the molecules that bind the notch region. Reagent can be added to stabilize the alpha helical character, such as trifluoroethanol. Reagents can also be added to increase the affinity between plastic and the notch region, such as a chemical immobilization through, for example, the amino terminus of the notch sequence-for example a COOH derivatized plastic could immobilize the notch peptide via carbodiimide activation and reaction with the lone amino group on the amino terminus of the notch peptide.

In other methods, a library of compounds can be dissolved at low concentration in micelles to mimic the membranous environment in which the viral notch normally functions. These solutions can be added to wells coated with the notch model compound, incubated to allow possible binding, then re-assayed to determine possible diminution in concentration.

In another example, molecules can also be identified using molecular modeling as discussed herein. Using the dimensions of the “notch”, approximately 5-6A deep and 10A wide a search of molecular structure databases, such as small molecule structure databases, to identify molecules that can bind the notch, such as small organic molecules, can be performed,. Hydrophobicity can also be added to the inquiry. Most “docking” programs usually assume an aqueous environment, the local dielectric can be set which could be set to mimic that of a membrane environment.

(1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed in=s one of any of the sequences disclosed herein or portions thereof, are used as the target in a combinatorial or screening protocol. It is understood that the physical dimensions as discussed herein of the notch can be used to design and implement a desired combinatorial type method.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, one of, for example, any of the sequences disclosed herein, are also disclosed. Thus, the products produced using the combinatorial or screening approaches that involve the disclosed compositions, one of, for example, one of any of the sequences disclosed herein, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars (oligosaccharides) are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind different small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that are attached to an acidic activation domain. A peptide of choice, for example a notch structural motif is attached to a DNA binding domain of a transcriptional activation protein, such as Gal 4. By performing the Two-hybrid technique on this type of system, molecules that bind the notch structural motif can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in iterative processes.

(2) Computer Assisted Identification

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, a notch structural motif domain are also disclosed. Thus, the products produced using the molecular modeling approaches that involve the disclosed compositions, such as, a notch structural motif, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other. Also a program called HINT has been used to examine interactions between the “notch” sequences of gp41 and CD4, as understood by the skilled artisan.

A number of articles review computer modeling of drugs interactive with specific proteins, such as Rotivinen, et al., 1988 Acta Phannaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

(a) Coordinates

Structure coordinates define a unique configuration of points in space. Those of skill in the art understand that a set of structure coordinates for protein or an protein/ligand complex, or a portion thereof, define a relative set of points that, in turn, define a configuration in three dimensions. A key piece of information obtained from the coordinates is the position of the atoms that make up the composition. The position of the atoms is defined in a Cartesian form, such that there are x-y-z positions which allow for a determination of distances and angles between two or more atoms. Thus, a similar or identical configuration, i.e. structure, can be defined by an entirely different set of coordinates, provided the distances and angles between coordinates remain essentially the same. By manipulating the distances and angles in a like manner a scalable representation can be obtained.

Disclosed are scalable three-dimensional configurations derived from structure coordinates, for example, set forth in Tables 3 and 4, or portion thereof, or from coordinates producing a configuration with essentially the same angles and distances between the atoms. Also disclosed are scalable three-dimensional configurations derived from the structure coordinates obtained from the disclosed molecules such as a notch structural motif. Other low energy structures can be produced using the disclosed coordinates as a starting point The data represented in Tables 3 and 4 were derived from performing standard calculations of the coordinates as disclosed herein. It is understood that once given the coordinate sets herein, the RMS (root mean square), for example, for any atom or subset of atoms can be calculated and is considered herein disclosed. Furthermore, it is understood that the various coordinates set forth in Tables 3 and 4 for any given individual atom represent a range for which that atom could take place in a coordinate representation of a notch structural motif or fragment thereof. Disclosed in Tables 3 and 4 are coordinates representing low energy structures of the complex of the notch structural motif and notch binding domain.

Also disclosed are scalable three-dimensional configurations of points derived from structure coordinates of molecules or molecular complexes that are structurally homologous to a notch structural motif and a notch binding domain, as well as structurally equivalent configurations, including the van der Waals surfaces.

The configurations of points in space derived from structure coordinates can be visualized as, for example, a holographic image, a stereodiagram, a model or a computer-displayed image, and the invention thus includes such images, diagrams or models.

Comparisons between different structures, different conformations of the same structure, and different parts of the same structure can be performed in a variety of ways. For example, typically the structures (coordinates making up the structure) are loaded, the atom equivalences in these structures are defined; the structures are fit, and then the resulting comparisons are reviewed.

Modeling programs typically also allow for a determination of the variances, the root mean square deviations, and statistical significance of the various structures.

The term “root mean square deviation” means the square root of the arithmetic mean of the squares of the deviations. This allows for comparison of two sets of data for example or the cognate position in two configurations or structures.

The tables disclosed herein that contain structure data follow the PDB format of the protein database. The formatting and nomenclature is that standard used throughout the industry.

(b) Hardware

The hardware architecture used for structural analysis and manipulation according to the present invention will include a system processor potentially including multiple processing elements where each processing element may be supported via a MIPS R10000 or R4400 processor such as provided in a SILICON GRAPHICS IMDIGO² IMPACT workstation; alternative processors such as Intel-compatible processor platforms using at least one PENTIUM III or CELERON (Intel Corp., Santa Clara, Calif.) class processor, UltraSPARC (Sun Microsystems, Palo Alto, Calif.) or other equivalent processors could be used in other embodiments. The system processor may include combinations of different processors from different vendors. In some embodiments, analysis and manipulation functionality, as further described below, may be distributed across multiple processing elements. The term processing element may refer to (1) a process running on a particular piece, or across particular pieces, of hardware, (2) a particular piece of hardware, or either (1) or (2) as the context allows.

The hardware includes a system data store (SDS) that could include a variety of primary and secondary storage elements. In one preferred embodiment, the SDS would include RAM as part of the primary storage; the amount of RAM might range from 32 MB to 640 MB although these amounts could vary and represent overlapping use. The primary storage may in some embodiments include other forms of memory such as cache memory, registers, non-volatile memory (e.g., FLASH, ROM, EPROM, etc.), etc.

The SDS may also include secondary storage including single, multiple and/or varied servers and storage elements. For example, the SDS may use internal storage devices connected to the system processor. In embodiments where a single processing element supports all of the analysis and manipulation functionality, a local hard disk drive may serve as the secondary storage of the SDS, and a disk operating system executing on such a single processing element may act as a data server receiving and servicing data requests.

The different information used in the processes and systems according to the present invention may be logically or physically segregated within a single device serving as secondary storage for the SDS; multiple related data stores accessible through a unified management system, which together serve as the SDS; or multiple independent data stores individually accessible through disparate management systems, which may in some embodiments be collectively viewed as the SDS. The various storage elements that comprise the physical architecture of the SDS may be centrally located, or distributed across a variety of diverse locations.

The architecture of the secondary storage of the system data store may vary significantly in different embodiments. In several embodiments, database(s) may be used to store and manipulate the data; in some such embodiments, one or more relational database management systems, such as DB2 (IBM, White Plains, N.Y.), SQL Server (Microsoft, Redmond, Wash.), ACCESS (Microsofi, Redmond, Wash.), ORACLE 8i (Oracle Corp., Redwood Shores, Calif.), Ingres (Computer Associates, Islandia, N.Y.), MySQL (MySQL AB, Sweden) or Adaptive Server Enterprise (Sybase Inc., Emeryville, Calif.), may be used in connection with a variety of storage devices/file servers that may include one or more standard magnetic and/or optical disk drives using any appropriate interface including, without limitation, IDE, EISA and SCSI. In some embodiments, a tape library such as Exabyte X80 (Exabyte Corporation, Boulder, Colo.), a storage attached network (SAN) solution such as available from (EMC, Inc., Hopkinton, Mass.), a network attached storage (NAS) solution such as a NetApp Filer 740 Network Appliances, Sunnyvale, Calif.), or combinations thereof may be used.

In other embodiments, the data store may use database systems with other architectures such as object-oriented, spatial, object-relational or hierarchical or may use other storage implementations such as hash tables or flat files or combinations of such architectures. Such alternative approaches may use data servers other than database management systems such as a hash table look-up server, procedure and/or process and/or a flat file retrieval server, procedure and/or process. Further, the SDS may use a combination of any of such approaches in organizing its secondary storage architecture.

In one preferred embodiment, coordinate data is stored in flat ASCII files according to a standardize format. In one such embodiment, the standardized format is PDB which is used through out the protein structure industry. The column content of the Tables containing coordinate data disclosed herein follows the PDB formatting and nomenclature.

The hardware platform would have an appropriate operating system such as WINDOWS/NT, WINDOWS 2000 or WINDOWS/XP Server (Microsoft, Redmond, Wash.), Solaris (Sun Microsystems, Palo Alto, Calif.), or IRIX (or other UNIX/LINUX variant). In one preferred embodiment, the hardware platform includes an IRIX operating system running on a SILICON GRAPHICS INDIGO² IMPACT workstation.

(c) Structural Coordinates and Storage of Same

Structural coordinates, such as atomic coordinates, of this invention can be stored in a machine-readable form on machine-readable storage medium. Examples of such media include, but are not limited to, computer hard drive, diskette, DAT tape, CD-ROM, and the like. The information stored on this media can be used for display as a three-dimensional shape or representation thereof or for other uses based on the structural coordinates, the spatial relationships between atoms described by the structural coordinates or the three-dimensional structures that they define. Such uses can include the use of a computer capable of reading the data from the storage media and executing instructions to generate and/or manipulate structures defined by the data. Commonly used sets of instructions, i.e., computer programs, for viewing or otherwise manipulating structures include, but are not limited to; Midas (UCSF), MidasPlus (UCSF), MOIL (University of Illinois), Yummie (Yale University), Sybyl (Tripos, Inc.), Insight/Discover (Biosym Technologies), MacroModel (Columbia University), Quanta (Molecular Simulations, Inc.), Cerius (Molucular Simulations, Inc.), Alchemy (Tripos, Inc.), LabVision (Tripos, Inc.), Rasmol (Glaxo Research and Development), Ribbon (University of Alabama), NAOMI (Oxford University), Explorer Eyechem (Silicon Graphics, Inc.), Univision (Cray Research), Molscript (Uppsala University), Chem-3D (Cambridge Scientific), Chain (Baylor College of Medicine), O (Uppsala University), GRASP (Columbia University), X-Plor (Molecular Simulations, Inc.; Yale University), Spartan (Wavefunction, Inc.), Catalyst (Molecular Simulations, Inc.), Molcadd (Tripos, Inc.), VMD (University of Illinois/Beckman Institute), Sculpt (Interactive Simulations, Inc.), Procheck (Brookhaven National Laboratory), DGEOM (QCPE), RE_VIEW (Brunel University), Modeller (Birbeck College, University of London), Xmol (Minnesota Supercomputing Center), Protein Expert (Cambridge Scientific), HyperChem (Hypercube), MD Display (University of Washington), PKB (National Center for Biotechnology Information, NIH), ChemX (Chemical Design, Ltd.), Cameleon (Oxford Molecular, Inc.), and Iditis (Oxford Molecular, Inc.).

(d) Machine Readable Storage Media

Disclosed are machine-readable storage mediums comprising a data storage material encoded with machine readable data. Furthermore, the data can be extracted and manipulated by machines configured to read the data stored on the machine readable storage media, and in fact, when performing the molecular modeling, such as displaying a configuration of the disclosed compositions, as discussed herein, typically the data will be retrieved or stored on a machine readable storage media.

Disclosed are machine readable storage media comprising the coordinates set forth in Table 3 and 4, or coordinates producing equivalent configurations of the disclosed compositions or their variants as discussed herein. Also disclosed are machine readable storage media comprising the coordinates set forth in Table 3 and 4 or a subset of these coordinates, or coordinates of any of coordinate tables disclosed herein or subsets of these, or coordinates producing equivalent configurations of the disclosed compositions or their variants as discussed herein.

Table 3 are representative coordinates full length 26 amino acid TM peptide containing a notch sequence (its from CD4_HUMAN)

TABLE 3

| | | | | | | | |
|---|---|---|---|---|---|---|---|
| ATOM | 1 | N | GLN | 1 | 0.000 | 1.335 | 0.000 |
| ATOM | 2 | H | GLN | 1 | 0.952 | 1.672 | −0.000 |
| ATOM | 3 | CA | GLN | 1 | −0.683 | 1.818 | 1.183 |
| ATOM | 4 | HA | GLN | 1 | −0.114 | 1.460 | 2.041 |
| ATOM | 5 | C | GLN | 1 | −2.110 | 1.291 | 1.246 |
| ATOM | 6 | O | GLN | 1 | −2.552 | 0.811 | 2.287 |
| ATOM | 7 | CB | GLN | 1 | −0.748 | 3.342 | 1.196 |
| ATOM | 8 | 1HB | GLN | 1 | 0.263 | 3.748 | 1.187 |
| ATOM | 9 | 2HB | GLN | 1 | −1.288 | 3.690 | 0.315 |
| ATOM | 10 | CG | GLN | 1 | −1.472 | 3.809 | 2.454 |
| ATOM | 11 | 1HG | GLN | 1 | −2.477 | 3.387 | 2.472 |
| ATOM | 12 | 2HG | GLN | 1 | −0.908 | 3.467 | 3.322 |
| ATOM | 13 | CD | GLN | 1 | −1.558 | 5.328 | 2.505 |
| ATOM | 14 | OE1 | GLN | 1 | −1.077 | 6.010 | 1.603 |
| ATOM | 15 | NE2 | GLN | 1 | −2.174 | 5.856 | 3.565 |
| ATOM | 16 | 1HE2 | GLN | 1 | −2.552 | 5.251 | 4.279 |
| ATOM | 17 | 2HE2 | GLN | 1 | −2.258 | 6.859 | 3.647 |
| ATOM | 18 | N | PRO | 2 | −2.839 | 1.379 | 0.128 |
| ATOM | 19 | CA | PRO | 2 | −4.211 | 0.903 | 0.091 |
| ATOM | 20 | HA | PRO | 2 | −4.718 | 1.181 | 1.014 |
| ATOM | 21 | C | PRO | 2 | −4.262 | −0.609 | −0.080 |
| ATOM | 22 | O | PRO | 2 | −4.995 | −1.293 | 0.631 |
| ATOM | 23 | CB | PRO | 2 | −4.930 | 1.540 | −1.062 |
| ATOM | 24 | 1HB | PRO | 2 | −5.284 | 0.765 | −1.742 |
| ATOM | 25 | 2HB | PRO | 2 | −5.779 | 2.111 | −0.688 |
| ATOM | 26 | CG | PRO | 2 | −3.987 | 2.462 | −1.796 |
| ATOM | 27 | 1HG | PRO | 2 | −3.859 | 2.111 | −2.820 |
| ATOM | 28 | 2HG | PRO | 2 | −4.365 | 3.484 | −1.828 |
| ATOM | 29 | CD | PRO | 2 | −2.677 | 2.377 | −1.071 |
| ATOM | 30 | 1HD | PRO | 2 | −2.408 | 3.362 | −0.689 |
| ATOM | 31 | 2HD | PRO | 2 | −1.894 | 2.030 | −1.746 |
| ATOM | 32 | N | MET | 3 | −3.478 | −1.130 | −1.027 |
| ATOM | 33 | H | MET | 3 | −2.898 | −0.514 | −1.578 |
| ATOM | 34 | CA | MET | 3 | −3.436 | −2.555 | −1.287 |
| ATOM | 35 | HA | MET | 3 | −4.438 | −2.846 | −1.603 |
| ATOM | 36 | C | MET | 3 | −3.037 | −3.329 | −0.038 |
| ATOM | 37 | O | MET | 3 | −3.670 | −4.324 | 0.308 |
| ATOM | 38 | CB | MET | 3 | −2.426 | −2.884 | −2.381 |
| ATOM | 39 | 1HB | MET | 3 | −2.707 | −2.370 | −3.301 |
| ATOM | 40 | 2HB | MET | 3 | −1.434 | −2.557 | −2.070 |
| ATOM | 41 | CG | MET | 3 | −2.413 | −4.389 | −2.625 |
| ATOM | 42 | 1HG | MET | 3 | −2.138 | −4.904 | −1.704 |
| ATOM | 43 | 2HG | MET | 3 | −3.406 | −4.709 | −2.941 |
| ATOM | 44 | SD | MET | 3 | −1.218 | −4.796 | −3.922 |
| ATOM | 45 | CE | MET | 3 | −1.418 | −6.564 | −3.984 |
| ATOM | 46 | 1HE | MET | 3 | −0.750 | −6.979 | −4.738 |
| ATOM | 47 | 2HE | MET | 3 | −1.177 | −6.991 | −3.010 |
| ATOM | 48 | 3HE | MET | 3 | −2.450 | −6.804 | −4.241 |
| ATOM | 49 | N | ALA | 4 | −1.983 | −2.868 | 0.639 |
| ATOM | 50 | H | ALA | 4 | −1.506 | −2.044 | 0.302 |
| ATOM | 51 | CA | ALA | 4 | −1.504 | −3.515 | 1.844 |
| ATOM | 52 | HA | ALA | 4 | −1.198 | −4.522 | 1.558 |
| ATOM | 53 | C | ALA | 4 | −2.597 | −3.582 | 2.901 |
| ATOM | 54 | O | ALA | 4 | −2.816 | −4.629 | 3.506 |
| ATOM | 55 | CB | ALA | 4 | −0.323 | −2.758 | 2.441 |
| ATOM | 56 | 1HB | ALA | 4 | 0.016 | −3.267 | 3.344 |
| ATOM | 57 | 2HB | ALA | 4 | 0.491 | −2.724 | 1.717 |
| ATOM | 58 | 3HB | ALA | 4 | −0.630 | −1.743 | 2.690 |
| ATOM | 59 | N | LEU | 5 | −3.283 | −2.459 | 3.123 |
| ATOM | 60 | H | LEU | 5 | −3.054 | −1.631 | 2.592 |
| ATOM | 61 | CA | LEU | 5 | −4.348 | −2.394 | 4.104 |
| ATOM | 62 | HA | LEU | 5 | −3.895 | −2.606 | 5.072 |
| ATOM | 63 | C | LEU | 5 | −5.436 | −3.414 | 3.801 |
| ATOM | 64 | O | LEU | 5 | −5.882 | −4.133 | 4.692 |
| ATOM | 65 | CB | LEU | 5 | −4.995 | −1.013 | 4.120 |
| ATOM | 66 | 1HB | LEU | 5 | −4.245 | −0.263 | 4.369 |
| ATOM | 67 | 2HB | LEU | 5 | −5.413 | −0.796 | 3.137 |
| ATOM | 68 | CG | LEU | 5 | −6.108 | −0.985 | 5.163 |
| ATOM | 69 | HG | LEU | 5 | −6.859 | −1.736 | 4.914 |
| ATOM | 70 | CD1 | LEU | 5 | −5.523 | −1.289 | 6.538 |
| ATOM | 71 | 1HD1 | LEU | 5 | −6.318 | −1.269 | 7.283 |
| ATOM | 72 | 2HD1 | LEU | 5 | −5.060 | −2.276 | 6.527 |
| ATOM | 73 | 3HD1 | LEU | 5 | −4.773 | −0.538 | 6.787 |
| ATOM | 74 | CD2 | LEU | 5 | −6.755 | 0.395 | 5.179 |
| ATOM | 75 | 1HD2 | LEU | 5 | −7.551 | 0.415 | 5.924 |
| ATOM | 76 | 2HD2 | LEU | 5 | −6.005 | 1.146 | 5.428 |
| ATOM | 77 | 3HD2 | LEU | 5 | −7.173 | 0.612 | 4.196 |
| ATOM | 78 | N | ILE | 6 | −5.863 | −3.475 | 2.537 |
| ATOM | 79 | H | ILE | 6 | −5.455 | −2.856 | 1.851 |
| ATOM | 80 | CA | ILE | 6 | −6.894 | −4.404 | 2.122 |
| ATOM | 81 | HA | ILE | 6 | −7.804 | −4.168 | 2.672 |
| ATOM | 82 | C | ILE | 6 | −6.491 | −5.841 | 2.424 |
| ATOM | 83 | O | ILE | 6 | −7.282 | −6.608 | 2.969 |
| ATOM | 84 | CB | ILE | 6 | −7.125 | −4.269 | 0.620 |
| ATOM | 85 | HB | ILE | 6 | −7.440 | −3.250 | 0.392 |
| ATOM | 86 | CG1 | ILE | 6 | −8.210 | −5.246 | 0.183 |
| ATOM | 87 | 1HG1 | ILE | 6 | −7.896 | −6.265 | 0.411 |
| ATOM | 88 | 2HG1 | ILE | 6 | −9.136 | −5.024 | 0.715 |
| ATOM | 89 | CG2 | ILE | 6 | −5.831 | −4.579 | −0.124 |
| ATOM | 90 | 1HG2 | ILE | 6 | −5.996 | −4.482 | −1.197 |
| ATOM | 91 | 2HG2 | ILE | 6 | −5.055 | −3.880 | 0.189 |
| ATOM | 92 | 3HG2 | ILE | 6 | −5.516 | −5.598 | 0.105 |
| ATOM | 93 | CD1 | ILE | 6 | −8.442 | −5.111 | −1.318 |
| ATOM | 94 | 1HD1 | ILE | 6 | −9.217 | −5.810 | −1.631 |
| ATOM | 95 | 2HD1 | ILE | 6 | −8.757 | −4.092 | −1.547 |
| ATOM | 96 | 3HD1 | ILE | 6 | −7.517 | −5.333 | −1.850 |
| ATOM | 97 | N | VAL | 7 | −5.257 | −6.203 | 2.069 |
| ATOM | 98 | H | VAL | 7 | −4.655 | −5.524 | 1.624 |
| ATOM | 99 | CA | VAL | 7 | −4.755 | −7.542 | 2.302 |
| ATOM | 100 | HA | VAL | 7 | −5.389 | −8.219 | 1.730 |
| ATOM | 101 | C | VAL | 7 | −4.811 | −7.898 | 3.781 |
| ATOM | 102 | O | VAL | 7 | −5.270 | −8.979 | 4.145 |
| ATOM | 103 | CB | VAL | 7 | −3.305 | −7.672 | 1.847 |
| ATOM | 104 | HB | VAL | 7 | −3.239 | −7.456 | 0.780 |
| ATOM | 105 | CG1 | VAL | 7 | −2.438 | −6.684 | 2.621 |
| ATOM | 106 | 1HG1 | VAL | 7 | −1.402 | −6.777 | 2.295 |
| ATOM | 107 | 2HG1 | VAL | 7 | −2.789 | −5.669 | 2.433 |
| ATOM | 108 | 3HG1 | VAL | 7 | −2.505 | −6.900 | 3.687 |
| ATOM | 109 | CG2 | VAL | 7 | −2.815 | −9.092 | 2.109 |
| ATOM | 110 | 1HG2 | VAL | 7 | −1.779 | −9.185 | 1.784 |
| ATOM | 111 | 2HG2 | VAL | 7 | −2.882 | −9.308 | 3.175 |
| ATOM | 112 | 3HG2 | VAL | 7 | −3.435 | −9.798 | 1.556 |
| ATOM | 113 | N | GLY | 8 | −4.343 | −6.984 | 4.634 |
| ATOM | 114 | H | GLY | 8 | −3.979 | −6.115 | 4.271 |
| ATOM | 115 | CA | GLY | 8 | −4.341 | −7.204 | 6.067 |
| ATOM | 116 | 1HA | GLY | 8 | −3.705 | −8.057 | 6.303 |
| ATOM | 117 | 2HA | GLY | 8 | −3.958 | −6.310 | 6.559 |
| ATOM | 118 | C | GLY | 8 | −5.754 | −7.471 | 6.564 |
| ATOM | 119 | O | GLY | 8 | −5.981 | −8.409 | 7.325 |
| ATOM | 120 | N | GLY | 9 | −6.707 | −6.643 | 6.130 |
| ATOM | 121 | H | GLY | 9 | −6.456 | −5.890 | 5.505 |
| ATOM | 122 | CA | GLY | 9 | −8.092 | −6.792 | 6.531 |
| ATOM | 123 | 1HA | GLY | 9 | −8.174 | −6.660 | 7.610 |
| ATOM | 124 | 2HA | GLY | 9 | −8.689 | −6.037 | 6.021 |
| ATOM | 125 | C | GLY | 9 | −8.610 | −8.171 | 6.148 |
| ATOM | 126 | O | GLY | 9 | −9.238 | −8.848 | 6.958 |
| ATOM | 127 | N | VAL | 10 | −8.344 | −8.585 | 4.907 |
| ATOM | 128 | H | VAL | 10 | −7.822 | −7.980 | 4.289 |
| ATOM | 129 | CA | VAL | 10 | −8.782 | −9.878 | 4.421 |
| ATOM | 130 | HA | VAL | 10 | −9.872 | −9.872 | 4.455 |
| ATOM | 131 | C | VAL | 10 | −8.238 | −11.003 | 5.289 |
| ATOM | 132 | O | VAL | 10 | −8.977 | −11.905 | 5.677 |
| ATOM | 133 | CB | VAL | 10 | −8.305 | −10.118 | 2.993 |
| ATOM | 134 | HB | VAL | 10 | −8.709 | −9.345 | 2.339 |
| ATOM | 135 | CG1 | VAL | 10 | −6.781 | −10.073 | 2.952 |
| ATOM | 136 | 1HG1 | VAL | 10 | −6.440 | −10.245 | 1.931 |
| ATOM | 137 | 2HG1 | VAL | 10 | −6.437 | −9.096 | 3.290 |
| ATOM | 138 | 3HG1 | VAL | 10 | −6.377 | −10.846 | 3.605 |
| ATOM | 139 | CG2 | VAL | 10 | −8.786 | −11.486 | 2.519 |
| ATOM | 140 | 1HG2 | VAL | 10 | −8.444 | −11.658 | 1.499 |
| ATOM | 141 | 2HG2 | VAL | 10 | −8.382 | −12.259 | 3.173 |
| ATOM | 142 | 3HG2 | VAL | 10 | −9.875 | −11.518 | 2.549 |
| ATOM | 143 | N | ALA | 11 | −6.939 | −10.948 | 5.594 |
| ATOM | 144 | H | ALA | 11 | −6.385 | −10.179 | 5.244 |
| ATOM | 145 | CA | ALA | 11 | −6.301 | −11.959 | 6.413 |
| ATOM | 146 | HA | ALA | 11 | −6.392 | −12.902 | 5.874 |
| ATOM | 147 | C | ALA | 11 | −6.975 | −12.067 | 7.773 |
| ATOM | 148 | O | ALA | 11 | −7.271 | −13.166 | 8.237 |
| ATOM | 149 | CB | ALA | 11 | −4.831 | −11.629 | 6.646 |
| ATOM | 150 | 1HB | ALA | 11 | −4.378 | −12.404 | 7.264 |
| ATOM | 151 | 2HB | ALA | 11 | −4.313 | −11.579 | 5.688 |
| ATOM | 152 | 3HB | ALA | 11 | −4.750 | −10.667 | 7.153 |
| ATOM | 153 | N | GLY | 12 | −7.217 | −10.921 | 8.414 |
| ATOM | 154 | H | GLY | 12 | −6.949 | −10.050 | 7.978 |
| ATOM | 155 | CA | GLY | 12 | −7.853 | −10.890 | 9.715 |
| ATOM | 156 | 1HA | GLY | 12 | −7.223 | −11.406 | 10.440 |
| ATOM | 157 | 2HA | GLY | 12 | −7.988 | −9.852 | 10.017 |
| ATOM | 158 | C | GLY | 12 | −9.216 | −11.566 | 9.655 |
| ATOM | 159 | O | GLY | 12 | −9.544 | −12.386 | 10.510 |
| ATOM | 160 | N | LEU | 13 | −10.011 | −11.218 | 8.641 |
| ATOM | 161 | H | LEU | 13 | −9.683 | −10.538 | 7.971 |
| ATOM | 162 | CA | LEU | 13 | −11.332 | −11.790 | 8.473 |
| ATOM | 163 | HA | LEU | 13 | −11.910 | −11.507 | 9.353 |
| ATOM | 164 | C | LEU | 13 | −11.263 | −13.306 | 8.360 |
| ATOM | 165 | O | LEU | 13 | −12.024 | −14.016 | 9.013 |
| ATOM | 166 | CB | LEU | 13 | −12.004 | −11.258 | 7.212 |
| ATOM | 167 | 1HB | LEU | 13 | −12.100 | −10.175 | 7.280 |
| ATOM | 168 | 2HB | LEU | 13 | −11.400 | −11.516 | 6.342 |
| ATOM | 169 | CG | LEU | 13 | −13.389 | −11.883 | 7.072 |
| ATOM | 170 | HG | LEU | 13 | −13.294 | −12.966 | 7.004 |
| ATOM | 171 | CD1 | LEU | 13 | −14.234 | −11.522 | 8.289 |
| ATOM | 172 | 1HD1 | LEU | 13 | −15.224 | −11.968 | 8.189 |
| ATOM | 173 | 2HD1 | LEU | 13 | −13.754 | −11.902 | 9.191 |
| ATOM | 174 | 3HD1 | LEU | 13 | −14.329 | −10.438 | 8.357 |
| ATOM | 175 | CD2 | LEU | 13 | −14.061 | −11.351 | 5.811 |
| ATOM | 176 | 1HD2 | LEU | 13 | −15.051 | −11.797 | 5.711 |
| ATOM | 177 | 2HD2 | LEU | 13 | −14.156 | −10.267 | 5.879 |
| ATOM | 178 | 3HD2 | LEU | 13 | −13.457 | −11.609 | 4.941 |
| ATOM | 179 | N | LEU | 14 | −10.346 | −13.802 | 7.526 |
| ATOM | 180 | H | LEU | 14 | −9.750 | −13.164 | 7.017 |
| ATOM | 181 | CA | LEU | 14 | −10.180 | −15.228 | 7.330 |
| ATOM | 182 | HA | LEU | 14 | −11.119 | −15.599 | 6.919 |
| ATOM | 183 | C | LEU | 14 | −9.872 | −15.930 | 8.645 |
| ATOM | 184 | O | LEU | 14 | −10.472 | −16.955 | 8.960 |
| ATOM | 185 | CB | LEU | 14 | −9.034 | −15.520 | 6.367 |
| ATOM | 186 | 1HB | LEU | 14 | −9.244 | −15.058 | 5.402 |
| ATOM | 187 | 2HB | LEU | 14 | −8.107 | −15.114 | 6.771 |
| ATOM | 188 | CG | LEU | 14 | −8.893 | −17.028 | 6.187 |
| ATOM | 189 | HG | LEU | 14 | −8.684 | −17.491 | 7.152 |
| ATOM | 190 | CD1 | LEU | 14 | −10.191 | −17.596 | 5.622 |
| ATOM | 191 | 1HD1 | LEU | 14 | −10.090 | −18.674 | 5.494 |
| ATOM | 192 | 2HD1 | LEU | 14 | −11.009 | −17.387 | 6.311 |
| ATOM | 193 | 3HD1 | LEU | 14 | −10.400 | −17.134 | 4.657 |
| ATOM | 194 | CD2 | LEU | 14 | −7.748 | −17.320 | 5.224 |
| ATOM | 195 | 1HD2 | LEU | 14 | −7.647 | −18.398 | 5.096 |
| ATOM | 196 | 2HD2 | LEU | 14 | −7.957 | −16.858 | 4.259 |
| ATOM | 197 | 3HD2 | LEU | 14 | −6.821 | −16.914 | 5.628 |
| ATOM | 198 | N | LEU | 15 | −8.934 | −15.373 | 9.414 |
| ATOM | 199 | H | LEU | 15 | −8.478 | −14.530 | 9.098 |
| ATOM | 200 | CA | LEU | 15 | −8.550 | −15.946 | 10.689 |
| ATOM | 201 | HA | LEU | 15 | −8.148 | −16.937 | 10.479 |
| ATOM | 202 | C | LEU | 15 | −9.747 | −16.055 | 11.623 |
| ATOM | 203 | O | LEU | 15 | −9.963 | −17.094 | 12.242 |
| ATOM | 204 | CB | LEU | 15 | −7.496 | −15.088 | 11.381 |
| ATOM | 205 | 1HB | LEU | 15 | −6.611 | −15.020 | 10.749 |
| ATOM | 206 | 2HB | LEU | 15 | −7.897 | −14.089 | 11.553 |
| ATOM | 207 | CG | LEU | 15 | −7.121 | −15.722 | 12.716 |
| ATOM | 208 | HG | LEU | 15 | −8.006 | −15.790 | 13.348 |
| ATOM | 209 | CD1 | LEU | 15 | −6.560 | −17.120 | 12.475 |
| ATOM | 210 | 1HD1 | LEU | 15 | −6.292 | −17.574 | 13.429 |
| ATOM | 211 | 2HD1 | LEU | 15 | −7.314 | −17.733 | 11.980 |
| ATOM | 212 | 3HD1 | LEU | 15 | −5.675 | −17.052 | 11.843 |
| ATOM | 213 | CD2 | LEU | 15 | −6.067 | −14.864 | 13.408 |
| ATOM | 214 | 1HD2 | LEU | 15 | −5.798 | −15.318 | 14.362 |
| ATOM | 215 | 2HD2 | LEU | 15 | −5.181 | −14.797 | 12.776 |
| ATOM | 216 | 3HD2 | LEU | 15 | −6.467 | −13.866 | 13.580 |
| ATOM | 217 | N | PHE | 16 | −10.528 | −14.976 | 11.723 |
| ATOM | 218 | H | PHE | 16 | −10.296 | −14.152 | 11.187 |
| ATOM | 219 | CA | PHE | 16 | −11.697 | −14.954 | 12.578 |
| ATOM | 220 | HA | PHE | 16 | −11.343 | −15.102 | 13.598 |
| ATOM | 221 | C | PHE | 16 | −12.674 | −16.058 | 12.199 |
| ATOM | 222 | O | PHE | 16 | −13.168 | −16.778 | 13.064 |
| ATOM | 223 | CB | PHE | 16 | −12.433 | −13.623 | 12.467 |
| ATOM | 224 | 1HB | PHE | 16 | −11.748 | −12.808 | 12.703 |
| ATOM | 225 | 2HB | PHE | 16 | −12.784 | −13.566 | 11.437 |
| ATOM | 226 | CG | PHE | 16 | −13.670 | −13.504 | 13.325 |
| ATOM | 227 | CD1 | PHE | 16 | −14.426 | −12.326 | 13.304 |
| ATOM | 228 | HD1 | PHE | 16 | −14.121 | −11.494 | 12.669 |
| ATOM | 229 | CD2 | PHE | 16 | −14.062 | −14.573 | 14.141 |
| ATOM | 230 | HD2 | PHE | 16 | −13.473 | −15.490 | 14.157 |
| ATOM | 231 | CE1 | PHE | 16 | −15.573 | −12.216 | 14.099 |
| ATOM | 232 | HE1 | PHE | 16 | −16.161 | −11.299 | 14.083 |
| ATOM | 233 | CE2 | PHE | 16 | −15.209 | −14.463 | 14.936 |
| ATOM | 234 | HE2 | PHE | 16 | −15.513 | −15.295 | 15.571 |
| ATOM | 235 | CZ | PHE | 16 | −15.964 | −13.284 | 14.915 |
| ATOM | 236 | HZ | PHE | 16 | −16.857 | −13.199 | 15.534 |
| ATOM | 237 | N | ILE | 17 | −12.952 | −16.191 | 10.900 |
| ATOM | 238 | H | ILE | 17 | −12.513 | −15.567 | 10.238 |
| ATOM | 239 | CA | ILE | 17 | −13.866 | −17.204 | 10.412 |
| ATOM | 240 | HA | ILE | 17 | −14.846 | −17.015 | 10.850 |
| ATOM | 241 | C | ILE | 17 | −13.405 | −18.597 | 10.815 |
| ATOM | 242 | O | ILE | 17 | −14.199 | −19.400 | 11.300 |
| ATOM | 243 | CB | ILE | 17 | −13.937 | −17.134 | 8.890 |
| ATOM | 244 | HB | ILE | 17 | −14.291 | −16.149 | 8.588 |
| ATOM | 245 | CG1 | ILE | 17 | −14.899 | −18.200 | 8.377 |
| ATOM | 246 | 1HG1 | ILE | 17 | −14.544 | −19.185 | 8.679 |
| ATOM | 247 | 2HG1 | ILE | 17 | −15.890 | −18.026 | 8.795 |
| ATOM | 248 | CG2 | ILE | 17 | −12.549 | −17.377 | 8.305 |
| ATOM | 249 | 1HG2 | ILE | 17 | −12.600 | −17.327 | 7.218 |
| ATOM | 250 | 2HG2 | ILE | 17 | −11.862 | −16.615 | 8.672 |
| ATOM | 251 | 3HG2 | ILE | 17 | −12.195 | −18.362 | 8.608 |
| ATOM | 252 | CD1 | ILE | 17 | −14.969 | −18.130 | 6.855 |
| ATOM | 253 | 1HD1 | ILE | 17 | −15.657 | −18.892 | 6.488 |
| ATOM | 254 | 2HD1 | ILE | 17 | −15.324 | −17.145 | 6.552 |
| ATOM | 255 | 3HD1 | ILE | 17 | −13.978 | −18.304 | 6.437 |
| ATOM | 256 | N | GLY | 18 | −12.117 | −18.883 | 10.611 |
| ATOM | 257 | H | GLY | 18 | −11.516 | −18.178 | 10.208 |
| ATOM | 258 | CA | GLY | 18 | −11.556 | −20.175 | 10.952 |
| ATOM | 259 | 1HA | GLY | 18 | −12.040 | −20.949 | 10.357 |
| ATOM | 260 | 2HA | GLY | 18 | −10.487 | −20.161 | 10.742 |
| ATOM | 261 | C | GLY | 18 | −11.763 | −20.469 | 12.431 |
| ATOM | 262 | O | GLY | 18 | −12.191 | −21.562 | 12.796 |
| ATOM | 263 | N | LEU | 19 | −11.456 | −19.488 | 13.284 |
| ATOM | 264 | H | LEU | 19 | −11.109 | −18.613 | 12.920 |
| ATOM | 265 | CA | LEU | 19 | −11.608 | −19.644 | 14.717 |
| ATOM | 266 | HA | LEU | 19 | −10.943 | −20.454 | 15.016 |
| ATOM | 267 | C | LEU | 19 | −13.046 | −19.988 | 15.081 |
| ATOM | 268 | O | LEU | 19 | −13.289 | −20.903 | 15.864 |
| ATOM | 269 | CB | LEU | 19 | −11.235 | −18.361 | 15.451 |
| ATOM | 270 | 1HB | LEU | 19 | −10.197 | −18.108 | 15.236 |
| ATOM | 271 | 2HB | LEU | 19 | −11.883 | −17.550 | 15.118 |
| ATOM | 272 | CG | LEU | 19 | −11.409 | −18.566 | 16.952 |
| ATOM | 273 | HG | LEU | 19 | −12.447 | −18.819 | 17.168 |
| ATOM | 274 | CD1 | LEU | 19 | −10.502 | −19.700 | 17.418 |
| ATOM | 275 | 1HD1 | LEU | 19 | −10.626 | −19.847 | 18.491 |
| ATOM | 276 | 2HD1 | LEU | 19 | −10.769 | −20.618 | 16.893 |
| ATOM | 277 | 3HD1 | LEU | 19 | −9.464 | −19.447 | 17.204 |
| ATOM | 278 | CD2 | LEU | 19 | −11.036 | −17.283 | 17.687 |
| ATOM | 279 | 1HD2 | LEU | 19 | −11.159 | −17.429 | 18.760 |
| ATOM | 280 | 2HD2 | LEU | 19 | −9.997 | −17.030 | 17.472 |
| ATOM | 281 | 3HD2 | LEU | 19 | −11.684 | −16.472 | 17.354 |
| ATOM | 282 | N | GLY | 20 | −14.000 | −19.250 | 14.509 |
| ATOM | 283 | H | GLY | 20 | −13.734 | −18.511 | 13.874 |
| ATOM | 284 | CA | GLY | 20 | −15.406 | −19.477 | 14.774 |
| ATOM | 285 | 1HA | GLY | 20 | −15.610 | −19.302 | 15.831 |
| ATOM | 286 | 2HA | GLY | 20 | −15.995 | −18.791 | 14.166 |
| ATOM | 287 | C | GLY | 20 | −15.790 | −20.905 | 14.414 |
| ATOM | 288 | O | GLY | 20 | −16.454 | −21.588 | 15.191 |
| ATOM | 289 | N | ILE | 21 | −15.368 | −21.357 | 13.230 |
| ATOM | 290 | H | ILE | 21 | −14.825 | −20.746 | 12.638 |
| ATOM | 291 | CA | ILE | 21 | −15.667 | −22.699 | 12.772 |
| ATOM | 292 | HA | ILE | 21 | −16.750 | −22.797 | 12.696 |
| ATOM | 293 | C | ILE | 21 | −15.145 | −23.741 | 13.750 |
| ATOM | 294 | O | ILE | 21 | −15.860 | −24.674 | 14.108 |
| ATOM | 295 | CB | ILE | 21 | −15.011 | −22.930 | 11.415 |
| ATOM | 296 | HB | ILE | 21 | −15.396 | −22.206 | 10.697 |
| ATOM | 297 | CG1 | ILE | 21 | −15.326 | −24.342 | 10.933 |
| ATOM | 298 | 1HG1 | ILE | 21 | −14.941 | −25.066 | 11.651 |
| ATOM | 299 | 2HG1 | ILE | 21 | −16.405 | −24.462 | 10.839 |
| ATOM | 300 | CG2 | ILE | 21 | −13.501 | −22.763 | 11.546 |
| ATOM | 301 | 1HG2 | ILE | 21 | −13.032 | −22.928 | 10.576 |
| ATOM | 302 | 2HG2 | ILE | 21 | −13.276 | −21.753 | 11.891 |
| ATOM | 303 | 3HG2 | ILE | 21 | −13.116 | −23.486 | 12.264 |
| ATOM | 304 | CD1 | ILE | 21 | −14.670 | −24.574 | 9.576 |
| ATOM | 305 | 1HD1 | ILE | 21 | −14.895 | −25.583 | 9.231 |
| ATOM | 306 | 2HD1 | ILE | 21 | −15.055 | −23.850 | 8.857 |
| ATOM | 307 | 3HD1 | ILE | 21 | −13.590 | −24.454 | 9.669 |
| ATOM | 308 | N | PHE | 22 | −13.892 | −23.580 | 14.182 |
| ATOM | 309 | H | PHE | 22 | −13.356 | −22.792 | 13.849 |
| ATOM | 310 | CA | PHE | 22 | −13.279 | −24.505 | 15.114 |
| ATOM | 311 | HA | PHE | 22 | −13.251 | −25.476 | 14.620 |
| ATOM | 312 | C | PHE | 22 | −14.083 | −24.598 | 16.403 |
| ATOM | 313 | O | PHE | 22 | −14.354 | −25.692 | 16.892 |
| ATOM | 314 | CB | PHE | 22 | −11.866 | −24.061 | 15.478 |
| ATOM | 315 | 1HB | PHE | 22 | −11.273 | −23.956 | 14.570 |
| ATOM | 316 | 2HB | PHE | 22 | −11.981 | −23.109 | 15.995 |
| ATOM | 317 | CG | PHE | 22 | −11.143 | −24.965 | 16.448 |
| ATOM | 318 | CD1 | PHE | 22 | −9.839 | −24.657 | 16.854 |
| ATOM | 319 | HD1 | PHE | 22 | −9.346 | −23.764 | 16.470 |
| ATOM | 320 | CD2 | PHE | 22 | −11.777 | −26.112 | 16.942 |
| ATOM | 321 | HD2 | PHE | 22 | −12.793 | −26.352 | 16.626 |
| ATOM | 322 | CE1 | PHE | 22 | −9.169 | −25.495 | 17.754 |
| ATOM | 323 | HE1 | PHE | 22 | −8.154 | −25.255 | 18.069 |
| ATOM | 324 | CE2 | PHE | 22 | −11.107 | −26.949 | 17.842 |
| ATOM | 325 | HE2 | PHE | 22 | −11.601 | −27.842 | 18.226 |
| ATOM | 326 | CZ | PHE | 22 | −9.803 | −26.641 | 18.247 |
| ATOM | 327 | HZ | PHE | 22 | −9.282 | −27.294 | 18.948 |
| ATOM | 328 | N | PHE | 23 | −14.466 | −23.443 | 16.953 |
| ATOM | 329 | H | PHE | 23 | −14.211 | −22.576 | 16.502 |
| ATOM | 330 | CA | PHE | 23 | −15.236 | −23.397 | 18.180 |
| ATOM | 331 | HA | PHE | 23 | −14.619 | −23.852 | 18.955 |
| ATOM | 332 | C | PHE | 23 | −16.542 | −24.165 | 18.035 |
| ATOM | 333 | O | PHE | 23 | −16.898 | −24.960 | 18.903 |
| ATOM | 334 | CB | PHE | 23 | −15.580 | −21.961 | 18.559 |
| ATOM | 335 | 1HB | PHE | 23 | −14.662 | −21.377 | 18.639 |
| ATOM | 336 | 2HB | PHE | 23 | −16.221 | −21.591 | 17.759 |
| ATOM | 337 | CG | PHE | 23 | −16.384 | −21.811 | 19.828 |
| ATOM | 338 | CD1 | PHE | 23 | −16.757 | −20.537 | 20.274 |
| ATOM | 339 | HD1 | PHE | 23 | −16.467 | −19.654 | 19.706 |
| ATOM | 340 | CD2 | PHE | 23 | −16.757 | −22.945 | 20.559 |
| ATOM | 341 | HD2 | PHE | 23 | −16.467 | −23.937 | 20.211 |
| ATOM | 342 | CE1 | PHE | 23 | −17.503 | −20.398 | 21.451 |
| ATOM | 343 | HE1 | PHE | 23 | −17.793 | −19.407 | 21.798 |
| ATOM | 344 | CE2 | PHE | 23 | −17.503 | −22.806 | 21.735 |
| ATOM | 345 | HE2 | PHE | 23 | −17.793 | −23.690 | 22.304 |
| ATOM | 346 | CZ | PHE | 23 | −17.876 | −21.533 | 22.181 |
| ATOM | 347 | HZ | PHE | 23 | −18.457 | −21.425 | 23.097 |
| ATOM | 348 | N | CYS | 24 | −17.258 | −23.926 | 16.934 |
| ATOM | 349 | H | CYS | 24 | −16.910 | −23.260 | 16.258 |
| ATOM | 350 | CA | CYS | 24 | −18.519 | −24.593 | 16.680 |
| ATOM | 351 | HA | CYS | 24 | −19.194 | −24.303 | 17.485 |
| ATOM | 352 | C | CYS | 24 | −18.345 | −26.105 | 16.661 |
| ATOM | 353 | O | CYS | 24 | −19.119 | −26.829 | 17.283 |
| ATOM | 354 | CB | CYS | 24 | −19.100 | −24.174 | 15.333 |
| ATOM | 355 | 1HB | CYS | 24 | −19.194 | −23.089 | 15.300 |
| ATOM | 356 | 2HB | CYS | 24 | −18.390 | −24.545 | 14.594 |
| ATOM | 357 | SG | CYS | 24 | −20.681 | −24.931 | 14.881 |
| ATOM | 358 | HG | CYS | 24 | −21.065 | −24.478 | 13.692 |
| ATOM | 359 | N | VAL | 25 | −17.323 | −26.580 | 15.945 |
| ATOM | 360 | H | VAL | 25 | −16.723 | −25.931 | 15.457 |
| ATOM | 361 | CA | VAL | 25 | −17.052 | −28.000 | 15.848 |
| ATOM | 362 | HA | VAL | 25 | −17.922 | −28.454 | 15.375 |
| ATOM | 363 | C | VAL | 25 | −16.827 | −28.610 | 17.225 |
| ATOM | 364 | O | VAL | 25 | −17.389 | −29.656 | 17.542 |
| ATOM | 365 | CB | VAL | 25 | −15.804 | −28.264 | 15.012 |
| ATOM | 366 | HB | VAL | 25 | −15.949 | −27.868 | 14.007 |
| ATOM | 367 | CG1 | VAL | 25 | −14.604 | −27.581 | 15.660 |
| ATOM | 368 | 1HG1 | VAL | 25 | −13.712 | −27.770 | 15.062 |
| ATOM | 369 | 2HG1 | VAL | 25 | −14.784 | −26.508 | 15.715 |
| ATOM | 370 | 3HG1 | VAL | 25 | −14.459 | −27.978 | 16.665 |
| ATOM | 371 | CG2 | VAL | 25 | −15.553 | −29.767 | 14.935 |
| ATOM | 372 | 1HG2 | VAL | 25 | −14.661 | −29.956 | 14.337 |
| ATOM | 373 | 2HG2 | VAL | 25 | −15.408 | −30.163 | 15.940 |
| ATOM | 374 | 3HG2 | VAL | 25 | −16.411 | −30.255 | 14.472 |
| ATOM | 375 | N | ARG | 26 | −16.002 | −27.953 | 18.043 |
| ATOM | 376 | H | ARG | 26 | −15.571 | −27.097 | 17.721 |
| ATOM | 377 | CA | ARG | 26 | −15.707 | −28.430 | 19.378 |
| ATOM | 378 | HA | ARG | 26 | −15.225 | −29.402 | 19.264 |
| ATOM | 379 | C | ARG | 26 | −16.978 | −28.571 | 20.203 |
| ATOM | 380 | O | ARG | 26 | −17.186 | −29.589 | 20.860 |
| ATOM | 381 | CB | ARG | 26 | −14.779 | −27.469 | 20.113 |
| ATOM | 382 | 1HB | ARG | 26 | −13.843 | −27.374 | 19.561 |
| ATOM | 383 | 2HB | ARG | 26 | −15.255 | −26.491 | 20.189 |
| ATOM | 384 | CG | ARG | 26 | −14.493 | −28.006 | 21.511 |
| ATOM | 385 | 1HG | ARG | 26 | −15.428 | −28.100 | 22.062 |
| ATOM | 386 | 2HG | ARG | 26 | −14.016 | −28.983 | 21.434 |
| ATOM | 387 | CD | ARG | 26 | −13.565 | −27.044 | 22.245 |
| ATOM | 388 | 1HD | ARG | 26 | −12.636 | −26.937 | 21.685 |
| ATOM | 389 | 2HD | ARG | 26 | −14.064 | −26.079 | 22.328 |
| ATOM | 390 | NE | ARG | 26 | −13.264 | −27.534 | 23.609 |
| ATOM | 391 | HE | ARG | 26 | −13.676 | −28.406 | 23.909 |
| ATOM | 392 | CZ | ARG | 26 | −12.477 | −26.879 | 24.457 |
| ATOM | 393 | NH1 | ARG | 26 | −11.899 | −25.725 | 24.135 |
| ATOM | 394 | 1HH1 | ARG | 26 | −12.055 | −25.324 | 23.221 |
| ATOM | 395 | 2HH1 | ARG | 26 | −11.307 | −25.256 | 24.805 |
| ATOM | 396 | NH2 | ARG | 26 | −12.275 | −27.411 | 25.659 |
| ATOM | 397 | 1HH2 | ARG | 26 | −12.715 | −28.287 | 25.901 |
| ATOM | 398 | 2HH2 | ARG | 26 | −11.682 | −26.936 | 26.325 |
| CONECT | 1 | 2 | 3 | | | | |
| CONECT | 2 | 1 | | | | | |
| CONECT | 3 | 1 | 4 | 5 | 7 | | |
| CONECT | 4 | 3 | | | | | |
| CONECT | 5 | 3 | 6 | 18 | | | |
| CONECT | 6 | 5 | | | | | |
| CONECT | 7 | 3 | 10 | 8 | 9 | | |
| CONECT | 8 | 7 | | | | | |
| CONECT | 9 | 7 | | | | | |
| CONECT | 10 | 7 | 13 | 11 | 12 | | |
| CONECT | 11 | 10 | | | | | |
| CONECT | 12 | 10 | | | | | |
| CONECT | 13 | 10 | 14 | 15 | | | |
| CONECT | 14 | 13 | | | | | |
| CONECT | 15 | 13 | 16 | 17 | | | |
| CONECT | 16 | 15 | | | | | |
| CONECT | 17 | 15 | | | | | |
| CONECT | 18 | 5 | 19 | 29 | | | |
| CONECT | 19 | 18 | 20 | 21 | 23 | | |
| CONECT | 20 | 19 | | | | | |
| CONECT | 21 | 19 | 22 | 32 | | | |
| CONECT | 22 | 21 | | | | | |
| CONECT | 23 | 19 | 26 | 24 | 25 | | |
| CONECT | 24 | 23 | | | | | |
| CONECT | 25 | 23 | | | | | |
| CONECT | 26 | 23 | 29 | 27 | 28 | | |
| CONECT | 27 | 26 | | | | | |
| CONECT | 28 | 26 | | | | | |
| CONECT | 29 | 18 | 26 | 30 | 31 | | |
| CONECT | 30 | 29 | | | | | |
| CONECT | 31 | 29 | | | | | |
| CONECT | 32 | 33 | 21 | 34 | | | |
| CONECT | 33 | 32 | | | | | |
| CONECT | 34 | 32 | 35 | 36 | 38 | | |
| CONECT | 35 | 34 | | | | | |
| CONECT | 36 | 34 | 37 | 49 | | | |
| CONECT | 37 | 36 | | | | | |
| CONECT | 38 | 34 | 41 | 39 | 40 | | |
| CONECT | 39 | 38 | | | | | |
| CONECT | 40 | 38 | | | | | |
| CONECT | 41 | 38 | 44 | 42 | 43 | | |
| CONECT | 42 | 41 | | | | | |
| CONECT | 43 | 41 | | | | | |
| CONECT | 44 | 41 | 45 | | | | |
| CONECT | 0 | 44 | | | | | |
| CONECT | 0 | 44 | | | | | |
| CONECT | 45 | 44 | 46 | 47 | 48 | | |
| CONECT | 46 | 45 | | | | | |
| CONECT | 47 | 45 | | | | | |
| CONECT | 48 | 45 | | | | | |
| CONECT | 49 | 50 | 36 | 51 | | | |
| CONECT | 50 | 49 | | | | | |
| CONECT | 51 | 49 | 52 | 53 | 55 | | |
| CONECT | 52 | 51 | | | | | |
| CONECT | 53 | 51 | 54 | 59 | | | |
| CONECT | 54 | 53 | | | | | |
| CONECT | 55 | 51 | 56 | 57 | 58 | | |
| CONECT | 56 | 55 | | | | | |
| CONECT | 57 | 55 | | | | | |
| CONECT | 58 | 55 | | | | | |
| CONECT | 59 | 60 | 53 | 61 | | | |
| CONECT | 60 | 59 | | | | | |
| CONECT | 61 | 59 | 62 | 63 | 65 | | |
| CONECT | 62 | 61 | | | | | |
| CONECT | 63 | 61 | 64 | 78 | | | |
| CONECT | 64 | 63 | | | | | |
| CONECT | 65 | 61 | 68 | 66 | 67 | | |
| CONECT | 66 | 65 | | | | | |
| CONECT | 67 | 65 | | | | | |
| CONECT | 68 | 65 | 69 | 70 | 74 | | |
| CONECT | 69 | 68 | | | | | |
| CONECT | 70 | 68 | 71 | 72 | 73 | | |
| CONECT | 71 | 70 | | | | | |
| CONECT | 72 | 70 | | | | | |
| CONECT | 73 | 70 | | | | | |
| CONECT | 74 | 68 | 75 | 76 | 77 | | |
| CONECT | 75 | 74 | | | | | |
| CONECT | 76 | 74 | | | | | |
| CONECT | 77 | 74 | | | | | |
| CONECT | 78 | 79 | 63 | 80 | | | |
| CONECT | 79 | 78 | | | | | |
| CONECT | 80 | 78 | 81 | 82 | 84 | | |
| CONECT | 81 | 80 | | | | | |
| CONECT | 82 | 80 | 83 | 97 | | | |
| CONECT | 83 | 82 | | | | | |
| CONECT | 84 | 80 | 86 | 89 | 85 | | |
| CONECT | 85 | 84 | | | | | |
| CONECT | 86 | 84 | 93 | 87 | 88 | | |
| CONECT | 87 | 86 | | | | | |
| CONECT | 88 | 86 | | | | | |
| CONECT | 89 | 84 | 90 | 91 | 92 | | |
| CONECT | 90 | 89 | | | | | |
| CONECT | 91 | 89 | | | | | |
| CONECT | 92 | 89 | | | | | |
| CONECT | 93 | 86 | 94 | 95 | 96 | | |
| CONECT | 94 | 93 | | | | | |
| CONECT | 95 | 93 | | | | | |
| CONECT | 96 | 93 | | | | | |
| CONECT | 97 | 98 | 82 | 99 | | | |
| CONECT | 98 | 97 | | | | | |
| CONECT | 99 | 97 | 100 | 101 | 103 | | |
| CONECT | 100 | 99 | | | | | |
| CONECT | 101 | 99 | 102 | 113 | | | |
| CONECT | 102 | 101 | | | | | |
| CONECT | 103 | 99 | 105 | 109 | 104 | | |
| CONECT | 104 | 103 | | | | | |
| CONECT | 105 | 103 | 106 | 107 | 108 | | |
| CONECT | 106 | 105 | | | | | |
| CONECT | 107 | 105 | | | | | |
| CONECT | 108 | 105 | | | | | |
| CONECT | 109 | 103 | 110 | 111 | 112 | | |
| CONECT | 110 | 109 | | | | | |
| CONECT | 111 | 109 | | | | | |
| CONECT | 112 | 109 | | | | | |
| CONECT | 113 | 114 | 101 | 115 | | | |
| CONECT | 114 | 113 | | | | | |
| CONECT | 115 | 113 | 116 | 117 | 118 | | |
| CONECT | 116 | 115 | | | | | |
| CONECT | 117 | 115 | | | | | |
| CONECT | 118 | 115 | 119 | 120 | | | |
| CONECT | 119 | 118 | | | | | |
| CONECT | 120 | 121 | 118 | 122 | | | |
| CONECT | 121 | 120 | | | | | |
| CONECT | 122 | 120 | 123 | 124 | 125 | | |
| CONECT | 123 | 122 | | | | | |
| CONECT | 124 | 122 | | | | | |
| CONECT | 125 | 122 | 126 | 127 | | | |
| CONECT | 126 | 125 | | | | | |
| CONECT | 127 | 128 | 125 | 129 | | | |
| CONECT | 128 | 127 | | | | | |
| CONECT | 129 | 127 | 130 | 131 | 133 | | |
| CONECT | 130 | 129 | | | | | |
| CONECT | 131 | 129 | 132 | 143 | | | |
| CONECT | 132 | 131 | | | | | |
| CONECT | 133 | 129 | 135 | 139 | 134 | | |
| CONECT | 134 | 133 | | | | | |
| CONECT | 135 | 133 | 136 | 137 | 138 | | |
| CONECT | 136 | 135 | | | | | |
| CONECT | 137 | 135 | | | | | |
| CONECT | 138 | 135 | | | | | |
| CONECT | 139 | 133 | 140 | 141 | 142 | | |
| CONECT | 140 | 139 | | | | | |
| CONECT | 141 | 139 | | | | | |
| CONECT | 142 | 139 | | | | | |
| CONECT | 143 | 144 | 131 | 145 | | | |
| CONECT | 144 | 143 | | | | | |
| CONECT | 145 | 143 | 146 | 147 | 149 | | |
| CONECT | 146 | 145 | | | | | |
| CONECT | 147 | 145 | 148 | 153 | | | |
| CONECT | 148 | 147 | | | | | |
| CONECT | 149 | 145 | 150 | 151 | 152 | | |
| CONECT | 150 | 149 | | | | | |
| CONECT | 151 | 149 | | | | | |
| CONECT | 152 | 149 | | | | | |
| CONECT | 153 | 154 | 147 | 155 | | | |
| CONECT | 154 | 153 | | | | | |
| CONECT | 155 | 153 | 156 | 157 | 158 | | |
| CONECT | 156 | 155 | | | | | |
| CONECT | 157 | 155 | | | | | |
| CONECT | 158 | 155 | 159 | 160 | | | |
| CONECT | 159 | 158 | | | | | |
| CONECT | 160 | 161 | 158 | 162 | | | |
| CONECT | 161 | 160 | | | | | |
| CONECT | 162 | 160 | 163 | 164 | 166 | | |
| CONECT | 163 | 162 | | | | | |
| CONECT | 164 | 162 | 165 | 179 | | | |
| CONECT | 165 | 164 | | | | | |
| CONECT | 166 | 162 | 169 | 167 | 168 | | |
| CONECT | 167 | 166 | | | | | |
| CONECT | 168 | 166 | | | | | |
| CONECT | 169 | 166 | 170 | 171 | 175 | | |
| CONECT | 170 | 169 | | | | | |
| CONECT | 171 | 169 | 172 | 173 | 174 | | |
| CONECT | 172 | 171 | | | | | |
| CONECT | 173 | 171 | | | | | |
| CONECT | 174 | 171 | | | | | |
| CONECT | 175 | 169 | 176 | 177 | 178 | | |
| CONECT | 176 | 175 | | | | | |
| CONECT | 177 | 175 | | | | | |
| CONECT | 178 | 175 | | | | | |
| CONECT | 179 | 180 | 164 | 181 | | | |
| CONECT | 180 | 179 | | | | | |
| CONECT | 181 | 179 | 182 | 183 | 185 | | |
| CONECT | 182 | 181 | | | | | |
| CONECT | 183 | 181 | 184 | 198 | | | |
| CONECT | 184 | 183 | | | | | |
| CONECT | 185 | 181 | 188 | 186 | 187 | | |
| CONECT | 186 | 185 | | | | | |
| CONECT | 187 | 185 | | | | | |
| CONECT | 188 | 185 | 189 | 190 | 194 | | |
| CONECT | 189 | 188 | | | | | |
| CONECT | 190 | 188 | 191 | 192 | 193 | | |
| CONECT | 191 | 190 | | | | | |
| CONECT | 192 | 190 | | | | | |
| CONECT | 193 | 190 | | | | | |
| CONECT | 194 | 188 | 195 | 196 | 197 | | |
| CONECT | 195 | 194 | | | | | |
| CONECT | 196 | 194 | | | | | |
| CONECT | 197 | 194 | | | | | |
| CONECT | 198 | 199 | 183 | 200 | | | |
| CONECT | 199 | 198 | | | | | |
| CONECT | 200 | 198 | 201 | 202 | 204 | | |
| CONECT | 201 | 200 | | | | | |
| CONECT | 202 | 200 | 203 | 217 | | | |
| CONECT | 203 | 202 | | | | | |
| CONECT | 204 | 200 | 207 | 205 | 206 | | |
| CONECT | 205 | 204 | | | | | |
| CONECT | 206 | 204 | | | | | |
| CONECT | 207 | 204 | 208 | 209 | 213 | | |
| CONECT | 208 | 207 | | | | | |
| CONECT | 209 | 207 | 210 | 211 | 212 | | |
| CONECT | 210 | 209 | | | | | |
| CONECT | 211 | 209 | | | | | |
| CONECT | 212 | 209 | | | | | |
| CONECT | 213 | 207 | 214 | 215 | 216 | | |
| CONECT | 214 | 213 | | | | | |
| CONECT | 215 | 213 | | | | | |
| CONECT | 216 | 213 | | | | | |
| CONECT | 217 | 218 | 202 | 219 | | | |
| CONECT | 218 | 217 | | | | | |
| CONECT | 219 | 217 | 220 | 221 | 223 | | |
| CONECT | 220 | 219 | | | | | |
| CONECT | 221 | 219 | 222 | 237 | | | |
| CONECT | 222 | 221 | | | | | |
| CONECT | 223 | 219 | 226 | 224 | 225 | | |
| CONECT | 224 | 223 | | | | | |
| CONECT | 225 | 223 | | | | | |
| CONECT | 226 | 223 | 227 | 229 | | | |
| CONECT | 227 | 226 | 231 | 228 | | | |
| CONECT | 228 | 227 | | | | | |
| CONECT | 229 | 226 | 233 | 230 | | | |
| CONECT | 230 | 229 | | | | | |
| CONECT | 231 | 227 | 235 | 232 | | | |
| CONECT | 232 | 231 | | | | | |
| CONECT | 233 | 229 | 235 | 234 | | | |
| CONECT | 234 | 233 | | | | | |
| CONECT | 235 | 231 | 233 | 236 | | | |
| CONECT | 236 | 235 | | | | | |
| CONECT | 237 | 238 | 221 | 239 | | | |
| CONECT | 238 | 237 | | | | | |
| CONECT | 239 | 237 | 240 | 241 | 243 | | |
| CONECT | 240 | 239 | | | | | |
| CONECT | 241 | 239 | 242 | 256 | | | |
| CONECT | 242 | 241 | | | | | |
| CONECT | 243 | 239 | 245 | 248 | 244 | | |
| CONECT | 244 | 243 | | | | | |
| CONECT | 245 | 243 | 252 | 246 | 247 | | |
| CONECT | 246 | 245 | | | | | |
| CONECT | 247 | 245 | | | | | |
| CONECT | 248 | 243 | 249 | 250 | 251 | | |
| CONECT | 249 | 248 | | | | | |
| CONECT | 250 | 248 | | | | | |
| CONECT | 251 | 248 | | | | | |
| CONECT | 252 | 245 | 253 | 254 | 255 | | |
| CONECT | 253 | 252 | | | | | |
| CONECT | 254 | 252 | | | | | |
| CONECT | 255 | 252 | | | | | |
| CONECT | 256 | 257 | 241 | 258 | | | |
| CONECT | 257 | 256 | | | | | |
| CONECT | 258 | 256 | 259 | 260 | 261 | | |
| CONECT | 259 | 258 | | | | | |
| CONECT | 260 | 258 | | | | | |
| CONECT | 261 | 258 | 262 | 263 | | | |
| CONECT | 262 | 261 | | | | | |
| CONECT | 263 | 264 | 261 | 265 | | | |
| CONECT | 264 | 263 | | | | | |
| CONECT | 265 | 263 | 266 | 267 | 269 | | |
| CONECT | 266 | 265 | | | | | |
| CONECT | 267 | 265 | 268 | 282 | | | |
| CONECT | 268 | 267 | | | | | |
| CONECT | 269 | 265 | 272 | 270 | 271 | | |
| CONECT | 270 | 269 | | | | | |
| CONECT | 271 | 269 | | | | | |
| CONECT | 272 | 269 | 273 | 274 | 278 | | |
| CONECT | 273 | 272 | | | | | |
| CONECT | 274 | 272 | 275 | 276 | 277 | | |
| CONECT | 275 | 274 | | | | | |
| CONECT | 276 | 274 | | | | | |
| CONECT | 277 | 274 | | | | | |
| CONECT | 278 | 272 | 279 | 280 | 281 | | |
| CONECT | 279 | 278 | | | | | |
| CONECT | 280 | 278 | | | | | |
| CONECT | 281 | 278 | | | | | |
| CONECT | 282 | 283 | 267 | 284 | | | |
| CONECT | 283 | 282 | | | | | |
| CONECT | 284 | 282 | 285 | 286 | 287 | | |
| CONECT | 285 | 284 | | | | | |
| CONECT | 286 | 284 | | | | | |
| CONECT | 287 | 284 | 288 | 289 | | | |
| CONECT | 288 | 287 | | | | | |
| CONECT | 289 | 290 | 287 | 291 | | | |
| CONECT | 290 | 289 | | | | | |
| CONECT | 291 | 289 | 292 | 293 | 295 | | |
| CONECT | 292 | 291 | | | | | |
| CONECT | 293 | 291 | 294 | 308 | | | |
| CONECT | 294 | 293 | | | | | |
| CONECT | 295 | 291 | 297 | 300 | 296 | | |
| CONECT | 296 | 295 | | | | | |
| CONECT | 297 | 295 | 304 | 298 | 299 | | |
| CONECT | 298 | 297 | | | | | |
| CONECT | 299 | 297 | | | | | |
| CONECT | 300 | 295 | 301 | 302 | 303 | | |
| CONECT | 301 | 300 | | | | | |
| CONECT | 302 | 300 | | | | | |
| CONECT | 303 | 300 | | | | | |
| CONECT | 304 | 297 | 305 | 306 | 307 | | |
| CONECT | 305 | 304 | | | | | |
| CONECT | 306 | 304 | | | | | |
| CONECT | 307 | 304 | | | | | |
| CONECT | 308 | 309 | 293 | 310 | | | |
| CONECT | 309 | 308 | | | | | |
| CONECT | 310 | 308 | 311 | 312 | 314 | | |
| CONECT | 311 | 310 | | | | | |
| CONECT | 312 | 310 | 313 | 328 | | | |
| CONECT | 313 | 312 | | | | | |
| CONECT | 314 | 310 | 317 | 315 | 316 | | |
| CONECT | 315 | 314 | | | | | |
| CONECT | 316 | 314 | | | | | |
| CONECT | 317 | 314 | 318 | 320 | | | |
| CONECT | 318 | 317 | 322 | 319 | | | |
| CONECT | 319 | 318 | | | | | |
| CONECT | 320 | 317 | 324 | 321 | | | |
| CONECT | 321 | 320 | | | | | |
| CONECT | 322 | 318 | 326 | 323 | | | |
| CONECT | 323 | 322 | | | | | |
| CONECT | 324 | 320 | 326 | 325 | | | |
| CONECT | 325 | 324 | | | | | |
| CONECT | 326 | 322 | 324 | 327 | | | |
| CONECT | 327 | 326 | | | | | |
| CONECT | 328 | 329 | 312 | 330 | | | |
| CONECT | 329 | 328 | | | | | |
| CONECT | 330 | 328 | 331 | 332 | 334 | | |
| CONECT | 331 | 330 | | | | | |
| CONECT | 332 | 330 | 333 | 348 | | | |
| CONECT | 333 | 332 | | | | | |
| CONECT | 334 | 330 | 337 | 335 | 336 | | |
| CONECT | 335 | 334 | | | | | |
| CONECT | 336 | 334 | | | | | |
| CONECT | 337 | 334 | 338 | 340 | | | |
| CONECT | 338 | 337 | 342 | 339 | | | |
| CONECT | 339 | 338 | | | | | |
| CONECT | 340 | 337 | 344 | 341 | | | |
| CONECT | 341 | 340 | | | | | |
| CONECT | 342 | 338 | 346 | 343 | | | |
| CONECT | 343 | 342 | | | | | |
| CONECT | 344 | 340 | 346 | 345 | | | |
| CONECT | 345 | 344 | | | | | |
| CONECT | 346 | 342 | 344 | 347 | | | |
| CONECT | 347 | 346 | | | | | |
| CONECT | 348 | 349 | 332 | 350 | | | |
| CONECT | 349 | 348 | | | | | |
| CONECT | 350 | 348 | 351 | 352 | 354 | | |
| CONECT | 351 | 350 | | | | | |
| CONECT | 352 | 350 | 353 | 359 | | | |
| CONECT | 353 | 352 | | | | | |
| CONECT | 354 | 350 | 357 | 355 | 356 | | |
| CONECT | 355 | 354 | | | | | |
| CONECT | 356 | 354 | | | | | |
| CONECT | 357 | 354 | 358 | | | | |
| CONECT | 358 | 357 | | | | | |
| CONECT | 0 | 357 | | | | | |
| CONECT | 0 | 357 | | | | | |
| CONECT | 359 | 360 | 352 | 361 | | | |
| CONECT | 360 | 359 | | | | | |
| CONECT | 361 | 359 | 362 | 363 | 365 | | |
| CONECT | 362 | 361 | | | | | |
| CONECT | 363 | 361 | 364 | 375 | | | |
| CONECT | 364 | 363 | | | | | |
| CONECT | 365 | 361 | 367 | 371 | 366 | | |
| CONECT | 366 | 365 | | | | | |
| CONECT | 367 | 365 | 368 | 369 | 370 | | |
| CONECT | 368 | 367 | | | | | |
| CONECT | 369 | 367 | | | | | |
| CONECT | 370 | 367 | | | | | |
| CONECT | 371 | 365 | 372 | 373 | 374 | | |
| CONECT | 372 | 371 | | | | | |
| CONECT | 373 | 371 | | | | | |
| CONECT | 374 | 371 | | | | | |
| CONECT | 375 | 376 | 363 | 377 | | | |
| CONECT | 376 | 375 | | | | | |
| CONECT | 377 | 375 | 378 | 379 | 381 | | |
| CONECT | 378 | 377 | | | | | |
| CONECT | 379 | 377 | 380 | | | | |
| CONECT | 380 | 379 | | | | | |
| CONECT | 381 | 377 | 384 | 382 | 383 | | |
| CONECT | 382 | 381 | | | | | |
| CONECT | 383 | 381 | | | | | |
| CONECT | 384 | 381 | 387 | 385 | 386 | | |
| CONECT | 385 | 384 | | | | | |
| CONECT | 386 | 384 | | | | | |
| CONECT | 387 | 384 | 390 | 388 | 389 | | |
| CONECT | 388 | 387 | | | | | |
| CONECT | 389 | 387 | | | | | |
| CONECT | 390 | 387 | 392 | 391 | | | |
| CONECT | 391 | 390 | | | | | |
| CONECT | 392 | 390 | 393 | 396 | | | |
| CONECT | 393 | 392 | 394 | 395 | | | |
| CONECT | 394 | 393 | | | | | |
| CONECT | 395 | 393 | | | | | |
| CONECT | 396 | 392 | 397 | 398 | | | |
| CONECT | 397 | 396 | | | | | |
| CONECT | 398 | 396 | | | | | |
| END | | | | | | | |

Table 4 are representative coordinates for a truncated HIV1 notch sequence from gp41

TABLE 4

| | | | | | | | |
|---|---|---|---|---|---|---|---|
| ATOM | 1 | N | ILE | 1 | 0.000 | 1.335 | 0.000 |
| ATOM | 2 | H | ILE | 1 | 0.952 | 1.672 | −0.000 |
| ATOM | 3 | CA | ILE | 1 | −0.683 | 1.818 | 1.183 |
| ATOM | 4 | HA | ILE | 1 | −0.137 | 1.465 | 2.058 |
| ATOM | 5 | C | ILE | 1 | −2.110 | 1.291 | 1.246 |
| ATOM | 6 | O | ILE | 1 | −2.552 | 0.811 | 2.287 |
| ATOM | 7 | CB | ILE | 1 | −0.727 | 3.342 | 1.158 |
| ATOM | 8 | HB | ILE | 1 | 0.290 | 3.735 | 1.140 |
| ATOM | 9 | CG1 | ILE | 1 | −1.446 | 3.850 | 2.403 |
| ATOM | 10 | 1HG1 | ILE | 1 | −2.462 | 3.458 | 2.422 |
| ATOM | 11 | 2HG1 | ILE | 1 | −0.911 | 3.517 | 3.293 |
| ATOM | 12 | CG2 | ILE | 1 | −1.474 | 3.809 | −0.086 |
| ATOM | 13 | 1HG2 | ILE | 1 | −1.505 | 4.898 | −0.104 |
| ATOM | 14 | 2HG2 | ILE | 1 | −0.960 | 3.446 | −0.976 |
| ATOM | 15 | 3HG2 | ILE | 1 | −2.491 | 3.417 | −0.068 |
| ATOM | 16 | CD1 | ILE | 1 | −1.489 | 5.375 | 2.379 |
| ATOM | 17 | 1HD1 | ILE | 1 | −2.003 | 5.738 | 3.269 |
| ATOM | 18 | 2HD1 | ILE | 1 | −0.472 | 5.767 | 2.360 |
| ATOM | 19 | 3HD1 | ILE | 1 | −2.023 | 5.708 | 1.489 |
| ATOM | 20 | N | VAL | 2 | −2.830 | 1.383 | 0.126 |
| ATOM | 21 | H | VAL | 2 | −2.408 | 1.788 | −0.697 |
| ATOM | 22 | CA | VAL | 2 | −4.201 | 0.917 | 0.056 |
| ATOM | 23 | HA | VAL | 2 | −4.770 | 1.512 | 0.770 |
| ATOM | 24 | C | VAL | 2 | −4.296 | −0.560 | 0.413 |
| ATOM | 25 | O | VAL | 2 | −5.151 | −0.957 | 1.202 |
| ATOM | 26 | CB | VAL | 2 | −4.771 | 1.095 | −1.347 |
| ATOM | 27 | HB | VAL | 2 | −4.748 | 2.150 | −1.617 |
| ATOM | 28 | CG1 | VAL | 2 | −3.934 | 0.297 | −2.341 |
| ATOM | 29 | 1HG1 | VAL | 2 | −4.341 | 0.424 | −3.343 |
| ATOM | 30 | 2HG1 | VAL | 2 | −2.904 | 0.655 | −2.319 |
| ATOM | 31 | 3HG1 | VAL | 2 | −3.957 | −0.759 | −2.070 |
| ATOM | 32 | CG2 | VAL | 2 | −6.211 | 0.594 | −1.377 |
| ATOM | 33 | 1HG2 | VAL | 2 | −6.619 | 0.721 | −2.380 |
| ATOM | 34 | 2HG2 | VAL | 2 | −6.234 | −0.462 | −1.107 |
| ATOM | 35 | 3HG2 | VAL | 2 | −6.809 | 1.164 | −0.667 |
| ATOM | 36 | N | GLY | 3 | −3.414 | −1.374 | −0.171 |
| ATOM | 37 | H | GLY | 3 | −2.736 | −0.985 | −0.810 |
| ATOM | 38 | CA | GLY | 3 | −3.401 | −2.800 | 0.087 |
| ATOM | 39 | 1HA | GLY | 3 | −4.343 | −3.237 | −0.245 |
| ATOM | 40 | 2HA | GLY | 3 | −2.572 | −3.249 | −0.461 |
| ATOM | 41 | C | GLY | 3 | −3.213 | −3.069 | 1.573 |
| ATOM | 42 | O | GLY | 3 | −3.930 | −3.879 | 2.156 |
| ATOM | 43 | N | GLY | 4 | −2.243 | −2.386 | 2.186 |
| ATOM | 44 | H | GLY | 4 | −1.688 | −1.735 | 1.650 |
| ATOM | 45 | CA | GLY | 4 | −1.964 | −2.553 | 3.598 |
| ATOM | 46 | 1HA | GLY | 4 | −1.650 | −3.580 | 3.787 |
| ATOM | 47 | 2HA | GLY | 4 | −1.169 | −1.865 | 3.883 |
| ATOM | 48 | C | GLY | 4 | −3.204 | −2.242 | 4.424 |
| ATOM | 49 | O | GLY | 4 | −3.562 | −3.000 | 5.323 |
| ATOM | 50 | N | VAL | 5 | −3.861 | −1.120 | 4.117 |
| ATOM | 51 | H | VAL | 5 | −3.515 | −0.540 | 3.367 |
| ATOM | 52 | CA | VAL | 5 | −5.055 | −0.713 | 4.829 |
| ATOM | 53 | HA | VAL | 5 | −4.762 | −0.556 | 5.868 |
| ATOM | 54 | C | VAL | 5 | −6.134 | −1.783 | 4.747 |
| ATOM | 55 | O | VAL | 5 | −6.742 | −2.134 | 5.756 |
| ATOM | 56 | CB | VAL | 5 | −5.629 | 0.574 | 4.247 |
| ATOM | 57 | HB | VAL | 5 | −4.889 | 1.370 | 4.324 |
| ATOM | 58 | CG1 | VAL | 5 | −5.987 | 0.353 | 2.781 |
| ATOM | 59 | 1HG1 | VAL | 5 | −6.398 | 1.272 | 2.365 |
| ATOM | 60 | 2HG1 | VAL | 5 | −5.092 | 0.071 | 2.227 |
| ATOM | 61 | 3HG1 | VAL | 5 | −6.728 | −0.444 | 2.704 |
| ATOM | 62 | CG2 | VAL | 5 | −6.882 | 0.968 | 5.022 |
| ATOM | 63 | 1HG2 | VAL | 5 | −7.293 | 1.888 | 4.606 |
| ATOM | 64 | 2HG2 | VAL | 5 | −7.622 | 0.172 | 4.945 |
| ATOM | 65 | 3HG2 | VAL | 5 | −6.626 | 1.126 | 6.070 |
| ATOM | 66 | N | ALA | 6 | −6.370 | −2.302 | 3.540 |
| ATOM | 67 | H | ALA | 6 | −5.836 | −1.970 | 2.750 |
| ATOM | 68 | CA | ALA | 6 | −7.372 | −3.328 | 3.331 |
| ATOM | 69 | HA | ALA | 6 | −8.331 | −2.890 | 3.608 |
| ATOM | 70 | C | ALA | 6 | −7.090 | −4.553 | 4.188 |
| ATOM | 71 | O | ALA | 6 | −7.989 | −5.078 | 4.842 |
| ATOM | 72 | CB | ALA | 6 | −7.403 | −3.776 | 1.873 |
| ATOM | 73 | 1HB | ALA | 6 | −8.164 | −4.546 | 1.746 |
| ATOM | 74 | 2HB | ALA | 6 | −7.638 | −2.924 | 1.236 |
| ATOM | 75 | 3HB | ALA | 6 | −6.428 | −4.179 | 1.597 |
| ATOM | 76 | N | GLY | 7 | −5.835 | −5.009 | 4.185 |
| ATOM | 77 | H | GLY | 7 | −5.142 | −4.532 | 3.626 |
| ATOM | 78 | CA | GLY | 7 | −5.439 | −6.168 | 4.959 |
| ATOM | 79 | 1HA | GLY | 7 | −5.982 | −7.044 | 4.606 |
| ATOM | 80 | 2HA | GLY | 7 | −4.367 | −6.323 | 4.837 |
| ATOM | 81 | C | GLY | 7 | −5.739 | −5.947 | 6.435 |
| ATOM | 82 | O | GLY | 7 | −6.303 | −6.817 | 7.094 |
| ATOM | 83 | N | LEU | 8 | −5.359 | −4.777 | 6.954 |
| ATOM | 84 | H | LEU | 8 | −4.900 | −4.102 | 6.358 |
| ATOM | 85 | CA | LEU | 8 | −5.588 | −4.446 | 8.346 |
| ATOM | 86 | HA | LEU | 8 | −5.032 | −5.174 | 8.936 |
| ATOM | 87 | C | LEU | 8 | −7.069 | −4.516 | 8.690 |
| ATOM | 88 | O | LEU | 8 | −7.447 | −5.102 | 9.702 |
| ATOM | 89 | CB | LEU | 8 | −5.103 | −3.035 | 8.662 |
| ATOM | 90 | 1HB | LEU | 8 | −4.034 | −2.964 | 8.457 |
| ATOM | 91 | 2HB | LEU | 8 | −5.640 | −2.318 | 8.040 |
| ATOM | 92 | CG | LEU | 8 | −5.361 | −2.726 | 10.132 |
| ATOM | 93 | HG | LEU | 8 | −6.429 | −2.797 | 10.337 |
| ATOM | 94 | CD1 | LEU | 8 | −4.609 | −3.728 | 11.002 |
| ATOM | 95 | 1HD1 | LEU | 8 | −4.793 | −3.508 | 12.053 |
| ATOM | 96 | 2HD1 | LEU | 8 | −4.956 | −4.737 | 10.776 |
| ATOM | 97 | 3HD1 | LEU | 8 | −3.541 | −3.657 | 10.797 |
| ATOM | 98 | CD2 | LEU | 8 | −4.875 | −1.315 | 10.448 |
| ATOM | 99 | 1HD2 | LEU | 8 | −5.060 | −1.094 | 11.500 |
| ATOM | 100 | 2HD2 | LEU | 8 | −3.807 | −1.244 | 10.244 |
| ATOM | 101 | 3HD2 | LEU | 8 | −5.413 | −0.599 | 9.827 |
| ATOM | 102 | N | ARG | 9 | −7.908 | −3.916 | 7.843 |
| ATOM | 103 | H | ARG | 9 | −7.534 | −3.451 | 7.028 |
| ATOM | 104 | CA | ARG | 9 | −9.341 | −3.913 | 8.059 |
| ATOM | 105 | HA | ARG | 9 | −9.515 | −3.388 | 8.998 |
| ATOM | 106 | C | ARG | 9 | −9.886 | −5.331 | 8.144 |
| ATOM | 107 | O | ARG | 9 | −10.660 | −5.649 | 9.045 |
| ATOM | 108 | CB | ARG | 9 | −10.066 | −3.203 | 6.920 |
| ATOM | 109 | 1HB | ARG | 9 | −9.721 | −2.171 | 6.857 |
| ATOM | 110 | 2HB | ARG | 9 | −9.857 | −3.715 | 5.981 |
| ATOM | 111 | CG | ARG | 9 | −11.568 | −3.221 | 7.184 |
| ATOM | 112 | 1HG | ARG | 9 | −11.914 | −4.253 | 7.248 |
| ATOM | 113 | 2HG | ARG | 9 | −11.778 | −2.709 | 8.124 |
| ATOM | 114 | CD | ARG | 9 | −12.293 | −2.511 | 6.046 |
| ATOM | 115 | 1HD | ARG | 9 | −11.935 | −1.484 | 5.971 |
| ATOM | 116 | 2HD | ARG | 9 | −12.086 | −3.046 | 5.119 |
| ATOM | 117 | NE | ARG | 9 | −13.756 | −2.509 | 6.269 |
| ATOM | 118 | HE | ARG | 9 | −14.118 | −2.950 | 7.102 |
| ATOM | 119 | CZ | ARG | 9 | −14.617 | −1.952 | 5.421 |
| ATOM | 120 | NH1 | ARG | 9 | −14.218 | −1.353 | 4.303 |
| ATOM | 121 | 1HH1 | ARG | 9 | −13.234 | −1.313 | 4.079 |
| ATOM | 122 | 2HH1 | ARG | 9 | −14.900 | −0.941 | 3.683 |
| ATOM | 123 | NH2 | ARG | 9 | −15.912 | −2.008 | 5.720 |
| ATOM | 124 | 1HH2 | ARG | 9 | −16.212 | −2.463 | 6.570 |
| ATOM | 125 | 2HH2 | ARG | 9 | −16.589 | −1.594 | 5.096 |
| CONECT | 1 | 2 | 3 | | | | |
| CONECT | 2 | 1 | | | | | |
| CONECT | 3 | 1 | 4 | 5 | 7 | | |
| CONECT | 4 | 3 | | | | | |
| CONECT | 5 | 3 | 6 | 20 | | | |
| CONECT | 6 | 5 | | | | | |
| CONECT | 7 | 3 | 9 | 12 | 8 | | |
| CONECT | 8 | 7 | | | | | |
| CONECT | 9 | 7 | 16 | 10 | 11 | | |
| CONECT | 10 | 9 | | | | | |
| CONECT | 11 | 9 | | | | | |
| CONECT | 12 | 7 | 13 | 14 | 15 | | |
| CONECT | 13 | 12 | | | | | |
| CONECT | 14 | 12 | | | | | |
| CONECT | 15 | 12 | | | | | |
| CONECT | 16 | 9 | 17 | 18 | 19 | | |
| CONECT | 17 | 16 | | | | | |
| CONECT | 18 | 16 | | | | | |
| CONECT | 19 | 16 | | | | | |
| CONECT | 20 | 21 | 5 | 22 | | | |
| CONECT | 21 | 20 | | | | | |
| CONECT | 22 | 20 | 23 | 24 | 26 | | |
| CONECT | 23 | 22 | | | | | |
| CONECT | 24 | 22 | 25 | 36 | | | |
| CONECT | 25 | 24 | | | | | |
| CONECT | 26 | 22 | 28 | 32 | 27 | | |
| CONECT | 27 | 26 | | | | | |
| CONECT | 28 | 26 | 29 | 30 | 31 | | |
| CONECT | 29 | 28 | | | | | |
| CONECT | 30 | 28 | | | | | |
| CONECT | 31 | 28 | | | | | |
| CONECT | 32 | 26 | 33 | 34 | 35 | | |
| CONECT | 33 | 32 | | | | | |
| CONECT | 34 | 32 | | | | | |
| CONECT | 35 | 32 | | | | | |
| CONECT | 36 | 37 | 24 | 38 | | | |
| CONECT | 37 | 36 | | | | | |
| CONECT | 38 | 36 | 39 | 40 | 41 | | |
| CONECT | 39 | 38 | | | | | |
| CONECT | 40 | 38 | | | | | |
| CONECT | 41 | 38 | 42 | 43 | | | |
| CONECT | 42 | 41 | | | | | |
| CONECT | 43 | 44 | 41 | 45 | | | |
| CONECT | 44 | 43 | | | | | |
| CONECT | 45 | 43 | 46 | 47 | 48 | | |
| CONECT | 46 | 45 | | | | | |
| CONECT | 47 | 45 | | | | | |
| CONECT | 48 | 45 | 49 | 50 | | | |
| CONECT | 49 | 48 | | | | | |
| CONECT | 50 | 51 | 48 | 52 | | | |
| CONECT | 51 | 50 | | | | | |
| CONECT | 52 | 50 | 53 | 54 | 56 | | |
| CONECT | 53 | 52 | | | | | |
| CONECT | 54 | 52 | 55 | 66 | | | |
| CONECT | 55 | 54 | | | | | |
| CONECT | 56 | 52 | 58 | 62 | 57 | | |
| CONECT | 57 | 56 | | | | | |
| CONECT | 58 | 56 | 59 | 60 | 61 | | |
| CONECT | 59 | 58 | | | | | |
| CONECT | 60 | 58 | | | | | |
| CONECT | 61 | 58 | | | | | |
| CONECT | 62 | 56 | 63 | 64 | 65 | | |
| CONECT | 63 | 62 | | | | | |
| CONECT | 64 | 62 | | | | | |
| CONECT | 65 | 62 | | | | | |
| CONECT | 66 | 67 | 54 | 68 | | | |
| CONECT | 67 | 66 | | | | | |
| CONECT | 68 | 66 | 69 | 70 | 72 | | |
| CONECT | 69 | 68 | | | | | |
| CONECT | 70 | 68 | 71 | 76 | | | |
| CONECT | 71 | 70 | | | | | |
| CONECT | 72 | 68 | 73 | 74 | 75 | | |
| CONECT | 73 | 72 | | | | | |
| CONECT | 74 | 72 | | | | | |
| CONECT | 75 | 72 | | | | | |
| CONECT | 76 | 77 | 70 | 78 | | | |
| CONECT | 77 | 76 | | | | | |
| CONECT | 78 | 76 | 79 | 80 | 81 | | |
| CONECT | 79 | 78 | | | | | |
| CONECT | 80 | 78 | | | | | |
| CONECT | 81 | 78 | 82 | 83 | | | |
| CONECT | 82 | 81 | | | | | |
| CONECT | 83 | 84 | 81 | 85 | | | |
| CONECT | 84 | 83 | | | | | |
| CONECT | 85 | 83 | 86 | 87 | 89 | | |
| CONECT | 86 | 85 | | | | | |
| CONECT | 87 | 85 | 88 | 102 | | | |
| CONECT | 88 | 87 | | | | | |
| CONECT | 89 | 85 | 92 | 90 | 91 | | |
| CONECT | 90 | 89 | | | | | |
| CONECT | 91 | 89 | | | | | |
| CONECT | 92 | 89 | 93 | 94 | 98 | | |
| CONECT | 93 | 92 | | | | | |
| CONECT | 94 | 92 | 95 | 96 | 97 | | |
| CONECT | 95 | 94 | | | | | |
| CONECT | 96 | 94 | | | | | |
| CONECT | 97 | 94 | | | | | |
| CONECT | 98 | 92 | 99 | 100 | 101 | | |
| CONECT | 99 | 98 | | | | | |
| CONECT | 100 | 98 | | | | | |
| CONECT | 101 | 98 | | | | | |
| CONECT | 102 | 103 | 87 | 104 | | | |
| CONECT | 103 | 102 | | | | | |
| CONECT | 104 | 102 | 105 | 106 | 108 | | |
| CONECT | 105 | 104 | | | | | |
| CONECT | 106 | 104 | 107 | | | | |
| CONECT | 107 | 106 | | | | | |
| CONECT | 108 | 104 | 111 | 109 | 110 | | |
| CONECT | 109 | 108 | | | | | |
| CONECT | 110 | 108 | | | | | |
| CONECT | 111 | 108 | 114 | 112 | 113 | | |
| CONECT | 112 | 111 | | | | | |
| CONECT | 113 | 111 | | | | | |
| CONECT | 114 | 111 | 117 | 115 | 116 | | |
| CONECT | 115 | 114 | | | | | |
| CONECT | 116 | 114 | | | | | |
| CONECT | 117 | 114 | 119 | 118 | | | |
| CONECT | 118 | 117 | | | | | |
| CONECT | 119 | 117 | 120 | 123 | | | |
| CONECT | 120 | 119 | 121 | 122 | | | |
| CONECT | 121 | 120 | | | | | |
| CONECT | 122 | 120 | | | | | |
| CONECT | 123 | 119 | 124 | 125 | | | |
| CONECT | 124 | 123 | | | | | |
| CONECT | 125 | 123 | | | | | |
| END | | | | | | | |

The disclosed coordinates and data can be manipulated on any appropriate machine, having for example, a processor, memory, and a monitor. The data can also be manipulated and accessed by a variety of connected items, including printers, LCDs, for example.

Disclosed are methods of utilizing molecular replacement to obtain structural information about a molecule or molecular complex whose structure is unknown comprising the steps of:

(a) producing coordinates of the molecule or molecular complex of unknown structure, and (b) applying at least a portion of the structure coordinates set forth in the disclosed coordinate tables to the coordinates of the unknown structure to generate a configuration of the unknown structure.

(e) Modeling of Variants

Structures of variant notch structural motifs, for example, can be produced without obtaining individual coordinates for the variant. In essence the coordinates of the molecules disclosed herein or coordinates that produce a structure homolog are used as a starting point and the variant atom or atoms of the variant disclosed molecule are substituted into the simulated structure and their relative position to the original unchanging atoms, i.e. coordinates, are determined through any of a variety of energy minimization functions. Thus, sequence alignment, secondary structure prediction, the screening of structural libraries of gp160, for example, or any of the other disclosed molecules, produced from the disclosed coordinates, or any combination of these can be used to overlay the variant structure. For example, the variant atom or atoms can also be modeled from any structural library having coordinates of similar or identical atoms. Thus, the initial structure to undergo energy minimization can be arrived at by modeling known coordinates for a given for the given atom or atoms. These libraries of structures can be screened for the optimal structure. A side chain rotomer library can be used to model a given side chain or set of side chains. After initial energy minimization iterative or new energy minimizations may be necessary if the structure produced after energy minimization violates a physical constraint, such as correct stereochemistry.

(f) Computer Drug Design

Computational techniques can be used to screen, identify, select and design chemical entities capable of associating with a notch structural motif, for example, or structurally homologous molecules, or complexes of the same. The disclosed coordinates and those that produce structurally homologous molecules can be used to model potential ligands for modulators, such as inhibitors, of CD4-gp120 interactions. Atoms of the potential ligand can be included in modeling simulation involving the notch structural motif, and other molecules as disclosed herein, and the contacts that arise between the potential ligand in a variety of positions with the disclosed compositions, or with a region, such as the CD4 notch binding domain, can be investigated. Energy minimization of these contacts between the potential ligand and the disclosed molecules can indicate potential ligands having, for example a desired affinity or a desired specificity. The ligands identified as having a desired number of contacts, with atoms of the disclosed compositions, such as the CD4-gp41 interaction mimix, as positioned by the coordinates or homologs disclosed herein, can be chosen and then optionally further tested by synthesizing or making the ligand and the disclosed compositions and performing standard biochemistry to assay binding activity or functional activity, such as those that use kinetic or thermodynamic methodology, such as, equilibrium dialysis, microcalorimetry, circular dichroism, capillary zone electrophoresis, nuclear magnetic resonance spectroscopy, fluorescence spectroscopy, and combinations thereof.

Drug designing typically involves computer-assisted design of chemical entities that associate with the notch structural motifs, their homologs, or portions thereof. Chemical entities can be designed in a step-wise fashion, one fragment at a time, or may be designed as a whole or “de novo.”

The binding sites of CD4 and gp160, such as the notch structural motif or the notch binding domain, as disclosed herein set forth the position of target atoms for interaction with ligands which will be able to bind or inhibit the disclosed interactions. The conformation of the notch structural motif and the notch structural motif binding site allow for a precise three dimensional map for rationally designing molecules that will form, for example, a set number of contacts with the atoms defining the binding regions as disclosed herein.

A contact as used herein means any position between two atoms, typically one atom of a ligand and one atom of the disclosed compositions, such as the notch structural motif or notch binding domain, that when positioned by an energy minimization program, for example, are less than 5A°, 4A°, 3A°, 2A°, or 1A° apart Thus, a contact can for example, correlate with, for example, non-covalent interactions, such as hydrogen bonds, Van der Waals interactions, hydrophobic interactions, and electrostatic interactions, between two atoms. Typically a contact will add to the binding energy between two atoms, but it can also be repulsive, typically more repulsive the closer the two atoms become. Although a contact is defined herein as being a relationship of two atoms, the molecules, components and compounds of which the atoms are a part can be referred to as having “contacts” with each other. Thus, for example, a ligand having an atom that forms a contact with an atom in a notch structural motif can be said to have a contact with the notch structural motif (and, more broadly, a contact with a protein comprising the notch structural motif). By further example, an inhibitor having an atom that forms a contact with an atom in an amino acid in a protein (such as gp160) can be said to have a contact with the amino acid in the protein. The contacts involved are the contacts between the atoms as descnbed above. It is understood that for a ligand to be a potential therapeutic candidate, it must have an appropriate level or quality of contacts, such that an interaction occurs, but that it should not cause steric and energetic problems. Typically there is a balance between favorable contacts and unfavorable contacts and in certain embodiments the balance is in favor of the favorable contacts to give the appropriate affinity. Conformational considerations include the overall three-dimensional structure and orientation of the chemical entity in relation to the binding pocket, and the spacing between various functional groups of an entity that directly interact with the notch structural motif or the notch binding domain or homologs thereof.

A contact between atoms, molecules, components or compounds is a form of interaction between the atom, molecules, components and compounds involved in the contact. Thus, an atom, molecule, component or compound can be said to “interact with” another atom, molecule, component or compound. Such an interaction can be referred to at any level. Thus, for example, an interaction (or contact) between two atoms in two different molecules results in a relationship between the two molecules that can be referred to as an interaction between the two molecules containing the atoms. Similarly, an interaction between, for example, an inhibitor and an amino acid of a protein results in a relationship between the inhibitor and the protein that can be referred to as an interaction between the inhibitor and the protein. Unless the context clearly indicates otherwise, reference to an interaction between atoms, molecules, components or compounds is not intended to exclude the existence of other, unstated interactions between the atoms, molecules, components or compounds at issue or with other atoms, molecules, components or compounds. Thus, for example, reference to an interaction between an inhibitor and one specific amino acid of a protein does not indicate that there are not other interactions or contacts between the inhibitor and the protein or with other atoms, molecules, components or compounds.

Unless the context clearly indicates otherwise, reference to the capability of atoms, molecules, components or compounds to interact with other atoms, molecules, components or compounds refers to the possibility of such an interaction should the atoms, molecules, components or compounds be brought into contact and not to any actual, presently existing interaction. Thus, for example, a statement that an inhibitor “can interact with” an amino acid of a protein refers to the fact that the inhibitor and amino acid would interact if brought into contact not that the inhibitor and amino acid are presently interacting.

The modeling and display of the disclosed compositions can be accomplished using any modeling program, such as QUANTA, SYBYL, CHARMM, and AMBER, Insight II/Discover (Molecular Simulations, Inc., San Diego, Calif. 92121); DelPhi (Molecular Simulations, Inc., San Diego, Calif. 92121); and AMSOL (Quantum Chemistry Program Exchange, Indiana University). These programs may be implemented, for example, using a Silicon Graphics workstation such as an Indigo² with “IMPACT” graphics. Other hardware systems and software packages will be known to those skilled in the art. Drug design programs, such as, GRID (P. J. Goodford, J. Med. Chem. 28:849-857 (1985); available from Oxford University, Oxford, UK); MCSS (A. Miranker et al., Proteins: Struct. Funct. Gen., 11:29-34 (1991); available from Molecular Simulations, San Diego, Calif.); AUTODOCK (D. S. Goodsell et al., Proteins: Struct. Funct. Genet. 8:195-202 (1990); available from Scripps Research Institute, La Jolla, Calif.); and DOCK (I. D. Kuntz et al., J. Mol. Biol. 161:269-288 (1982); available from University of California, San Francisco, Calif.), LUDI (H.-J. Bohm, J. Comp. Aid. Molec. Design. 6:61-78 (1992); available from Molecular Simulations Inc., San Diego, Calif.); LEGEND (Y. Nishibata et al., Tetrahedron, 47:8985 (1991); available from Molecular Simulations Inc., San Diego, Calif.); LeapFrog (available from Tripos Associates, St Louis, Mo.); and SPROUT (V. Gillet et al., J. Comput. Aided Mol. Design 7:127-153 (1993); available from the University of Leeds, UK), can also be used.

The efficiency of a potential ligand's interaction with the disclosed compositions can be evaluated and optimized. For example, typically a preferred ligand will cause little perturbation to the three dimensional positioning of the atoms of disclosed compositions that are in the vicinity of the interaction or are somehow allosterically affected. The level of perturbation can be determined by comparing the energy state of the disclosed structural conformations for the bound and unbound states. Typically the smaller the change the less perturbation and the less perturbation the higher the likelihood that the ligand will be desirable as for example, a competitive inhibitor. This perturbation energy can be, for example, less than or equal to about 30 kcal/mole, 20 kcal/mole, 15 kcal/mole, 10 kcal/mole, 8 kcal/mole, 6 kcal/mole, 5 kcal/mole, 4 kcal/mole, 3 kcal/mole, 2 kcal/mole, or 1 kcal/mole. Notch structural motif or notch binding domain ligands may interact with the gp160 or CD4 molecule in more than one conformation that is similar in overall binding energy. In those cases, the perturbation energy of binding can be taken as the difference between the energy of the free entity and the average energy of the conformations observed when the ligand binds to the gp160 or CD4 or notch structural motif or notch binding domain.

An entity designed or selected as binding to a notch structural motif or notch binding domain may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. 15106); AMBER, version 4.1 (P. A. Kollman, University of California at San Francisco, 94143); QUANTA/CHARMM (Molecular Simulations, Inc., San Diego, Calif. 92121);

The disclosed structures and coordinates can also be used to screen potential ligands, for example, as drug, candidates, which interact with, i.e. form contacts with, the notch binding domain or notch structural motif Small molecule databases, such as structure databases can be used for this. Not only whole molecules can be screened, but subparts of molecule, for example, various functional groups can also be screen to find preferred functional groups for forming contacts with the notch structural motif or notch binding domain structures disclosed herein. Functional groups that make a desired set of contacts, for example, with a desired or particular region of the notch structural motif or notch binding domain, can then be used to further build combinations of these and other types of functional groups to design ligands containing the functional groups or combinations of functional groups.

It is understood that also disclosed are iterative approaches which use successive performance of the various steps disclosed herein to optimize molecules and/or isolate molecules from sets of molecules. This can also be done with multiple coordinate sets that have been obtained, for example, from the solution of structures involving a ligand or series of structures involving a series of ligands. For example, molecules known to have preferred biochemical properties, such as binding the notch structural motif or notch binding domain as disclosed herein, can be solved in a co-structure, and then the structure information obtained from this can be used to select potential ligands for function.

A compound that is identified or designed as a result of any of these methods can be obtained (or synthesized) and tested for its biological activity, e.g., inhibition of CD4-gp160 interaction activity.

Also disclosed are scalable three dimensional sets of points derived from structure coordinates of at least a portion of a molecule or a molecular complex that is structurally homologous to a notch structural motif or a notch binding domain optionally including their complexes. Two points are considered structurally homologous if they have RMS of less than 5 A°, 4 A°, 3 A°, 2 A°., or 1.0A°. A structurally homologous structure would have an average of less than 5 A°, 4 A°, 3 A°, 2 A°., or 1.0A° RMS.

An analog structure is a structure that has a different chemical make up, but which has a homologous structure to the reference structure, such as a structure of a notch structural motif or a notch binding domain.

Although described above with reference to design and generation of compounds which could alter binding, for example, to the notch, or inhibit notch function, one could also screen libraries of known compounds, including natural products or synthetic chemicals, and biologically active materials, including proteins, for compounds which alter substrate binding or HIV infectivity, for example. For example, biotin can be added to a notch sequence, such as SEQ ID NO:6. This molecule can then be incubated with, for example, disrupted T cell membranes. The mixture can collected on a column that can react with biotin, such as streptavidin, or an anti-biotin-antibody. The column can then be washed, for example, with a neutral pH solution, and then bound molecules can be collected, by for example, a low pH solution or heating. The collected molecules, can, for example, be analyzed by other chromatographic methods, such as SDS-PAGE or HPLC. Identified molecules, can be further analyzed, for example, by using the peptide-biotin conjugate in a Western-type blot developed by streptavidin-peroxidase. Control and comparative samples, may include membranes lacking CD4. This type of assay can also be used with known inhibitors and interactorsaThe samples might—as control—include membranes lacking CD4. Candidate known molecules such as synthetic CD4 peptides can be examined too. One requirement for us would be to do this in a solvent that reproduces the presumed membranous environ.

Molecules that bind the notch region can be identified. As disclosed herein the notch region is related to the helical domain as set forth in for example, SEQ ID NOs: 1 and 2, for example.

The disclosed methods can use energy transfer donor and acceptor molecule pairs to identify notch inhibitors in high through-put assays. For example, a molecule comprising a notch region can be associated with an energy transfer donor. Another molecule comprising a notch region can be associated with an energy transfer acceptor and these molecules can then be incubated together. When the acceptor notch region and donor notch region interact there will be an increase of the fluorescence (RET [resonance energy transfer]). Molecules which are able to compete the notch-notch interaction will reduce this fluorescence, and can be identified on this basis.

3. Characteristics of Compositions

a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid or protein sequences. Many of the methods for determining homology between two evolutionarily related molecules are routinely applied to any two or more nucleic acids or proteins for the purpose of measuring sequence similarity regardless of whether they are evolutionarily related or not.

In general, itis understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence, but in many cases can be as low as 10, 15, 20, 25, 30, 35, 40, 55, 60, or 65% homology because the requirement sequences with very low homologies can still form helical notch sequences. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized inplementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example notch structural motifs or molecules that bind notch structural motifs, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙ O]ₘ CH₃, —O(CH₂)ₙ OCH₃, —O(CH₂)ₙ NH₂, —O(CH₂)ₙ CH₃, —O(CH₂)ₙ —ONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacolinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manobaran et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to the the CD4 and gp160 gene having the following Genbank Accession Numbers as disclosed herein these sequences and others are herein incorporated by reference in their entireties as well as for individual subsequences contained therein.

One particular sequence set forth in SEQ ID NO:26 and used herein, as an example, to exemplify the disclosed compositions and methods. It is understood that the description related to this sequence is applicable to any sequence related to SEQ ID NO:26 unless specifically indicated otherwise. Those of skill in the art understand how to resolve sequence discrepancies and differences and to adjust the compositions and methods relating to a particular sequence to other related sequences (i.e. sequences of CD4 or gp160, for example). Primers and/or probes can be designed for any CD4 or gp160 sequence given the information disclosed herein and known in the art.

d) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as those encoding notch structural motifs or molecules that bind notch structural motifs, into the cell without degradation and include a promoter yielding expression of the gene in the cells into which it is delivered. In some embodiments the vectors are derived from either a DNA virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the basic HIV framework. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

(c) Adeno-associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated SP by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(d) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson,. Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

(2) Non-nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of delivery, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In vivo/ex vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject

e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(1) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elenients can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells yeast, fungi, insect, plant, animal, human or nucleated cells) may also contain sequences necessary for the termination of transcription which may affect mRNA expression. These regions are transcribed as polyadenylated segments in the untranslated portion of the rnRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media Two examples are: CHO DHFR- cells and mouse LTK- cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

f) Peptides

(1) Protein Variants

As discussed herein there are numerous variants of the notch structural motifs and related proteins, such as gp160 and CD4, that are known and herein contemplated. In addition to the known functional gp160 strain variants and other variants there are derivatives of the notch structural motifs, for example, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for maling substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

216.

TABLE 1
Amino Acid Abbreviations

| | |
|---|---|
| Amino Acid | Abbreviations |

| | | |
|---|---|---|
| Alanine | Ala | A |

| | |
|---|---|
| Allosoleucine | AIle |

| | | |
|---|---|---|
| Arginine | Arg | R |
| Asparagines | Asn | N |
| aspartic acid | Asp | D |
| Cysteine | Cys | C |
| glutamic acid | Glu | E |
| Glutamine | Gln | K |
| Glycine | Gly | G |
| Histidine | His | H |
| Isolelucine | Ile | I |
| Leucine | Leu | L |
| Lysine | Lys | K |
| Phenylalanine | Phe | F |
| Proline | Pro | P |

| | |
|---|---|
| pyroglutamic acid | Glup |

| | | |
|---|---|---|
| Serine | Ser | S |
| Threonine | Thr | T |
| Tyrosine | Tyr | Y |
| tryptophan | Trp | W |
| Valine | Val | V |

TABLE 2
Amino Acid Substitutions
Original Residue Exemplary Conservative Substitutions,
others are known in the art.

| | |
|---|---|
| Ala | gly. ser |
| Ar | glys, gln |
| Asn | gln; his |
| Asp | glu |
| Cys | ser |
| Gln | asn, lys |
| Glu | asp |
| Gly | ala, pro depending upon whether the gly plays |
| | a packing role [ala] or a turn role [pro] |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl or tyrosyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:1 sets forth a particular sequence of a notch structural motif. Specifically disclosed are variants of these and other proteins herein disclosed which have at least, 10% or 15% or 20% or 25% or 30% or 35% or 40% or 45% or 50% or 60% or 65% or 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:26 is set forth in SEQ ID NO:27. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent than the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs). Chemical synthesis of peptides containing d-amino acids can also be readily accomplished, and for example, peptides containing all d-amino acids can be made, by methods well known in the art.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO-(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

g) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Formulations for topical administration may include transdermal patches. Coated condoms, gloves and the like may also be useful.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Compositions for parenteral, intrathecal or intraventricular administration may include sterile aqueous solutions which may also contain buffers, diluents and other suitable additives.

In addition to such pharmaceutical carriers, cationic lipids may be included in the formulation to facilitate uptake. One such composition shown to facilitate uptake is Lipofectin (BRL, Bethesda Md.).

(2) Therapeutic Uses

Disclosed are methods of decreasing interaction of human immunodeficiency virus with a host cell. Effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Dosing is dependent on severity and responsiveness of the condition to be treated, with course of treatment lasting from several days to several months or until a cure is effected or a diminution of disease state is achieved. In the case of a healthy subject, course of treatment can last as long as there is a risk of exposure.

Optimal dosing schedules can be calculated from measurements of drug accumulation in the body. The optimum dosages can be determined using dosing methodologies and repetition rates. Optimum dosages may vary depending on the relative potency of individual compositions, and can generally be calculated based on IC₅₀'s or EC₅₀'s in in vitro and in vivo animal studies. For example, given the molecular weight of compound and an effective dose such as an IC₅₀, for example (derived experimentally), a dose in mg/kg is routinely calculated.

Following administration of a disclosed composition, such as an antibody or peptide, for treating, inhibiting, or preventing an HIV infection, the efficacy of the therapeutic antibody can be assessed in various ways well known to the skilled practitioner. For instance, one of ordinary skill in the art will understand that a composition, such as an antibody, disclosed herein is efficacious in treating or inhibiting an HIV infection in a subject by observing that the composition reduces viral load or prevents a further increase in viral load. Viral loads can be measured by methods that are known in the art, for example, using polymerase chain reaction assays to detect the presence of HIV nucleic acid or antibody assays to detect the presence of HIV protein in a sample (e.g., but not limited to, blood) from a subject or patient, or by measuring the level of circulating anti-HIV antibody levels in the patient. Efficacy of the administration of the disclosed composition may also be determined by measuring the number of CD4⁺ T cells in the HIV-infected subject An antibody treatment that inhibits an initial or further decrease in CD4⁺ T cells in an HIV-positive subject or patient, or that results in an increase in the number of CD4⁺ T cells in the HIV-positive subject, is an efficacious antibody treatment.

The compositions that inhibit CD4-gp160 interactions disclosed herein maybe administered prophylactically to patients or subjects who are at risk for HIV infection, such as being exposed to HIV or who have been newly exposed to HIV. In subjects who have been newly exposed to HIV but who have not yet displayed the presence of the virus (as measured by PCR or other assays for detecting the virus) in blood or other body fluid, efficacious treatment with an antibody partially or completely inhibits the appearance of the virus in the blood or other body fluid.

Other molecules that interact with notch domains or notch binding domains to inhibit CD4-gp160 interactions which do not have a specific pharmacuetical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of HIV related disorders.

Molecules capable of interfering with binding of a target within glycoprotein 160 of HIV-1 to a putative host cell ligand for the target, tissues or cells could be contacted with compositions of the molecules in order to decrease interaction of human immunodeficiency virus with a host cell. “Contact” tissues or cells with a composition means to add the composition, usually in a suitable liquid carrier, to a cell suspension or tissue sample, either in vitro or ex vivo, or to administer the composition to cells or tissues within an animal (including humans). By contacting the tissues or cells with the compositions of the molecules, the gp 160 protein and/or the ligand present in the tissues or cells is thereby exposed to the molecule.

4. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

5. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

C. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:1, is to link two or more peptides or polypeptides or amino acids together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton RC et al., Techniques in Protein Chemistry IV. Academic Press, New York, pp. 257-267 (1992)).

3. Methods of Making Cells and Animals

Disclosed are cells produced by the process of transforming the cell with any of the disclosed nucleic acids or peptides. Disclosed are cells produced by the process of contacting the cell with any of the non-naturally occurring disclosed nucleic acids or peptides.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

D. Methods of Using the Compositions

1. Methods of Using the Compositions as Research Tools

The disclosed compositions can be used in a variety of ways as research tools. For example, the disclosed compositions, such as SEQ ID NOs:1-25 can be used to study the interactions between CD4 and gp160, by for example acting as inhibitors of binding.

The compositions can be used for example as targets in combinatorial chemistry protocols or other screening protocols to isolate molecules that possess desired functional properties related to CD4 and gp160 binding.

The disclosed compositions can also be used diagnostic tools related to diseases, such as HIV, by for example, identifying the presence of a notch sequence in an HIV isolate.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining strain analysis of for example, HIV isolates. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Materials and Methods.

(1) Sequence Comparisons.

Initially, sequences conserved within gp41, particularly within the TM domains, were identified using the PC/GENE programs PALIGN and CLUSTAL. Then, potential sequence similarities between CD4 and gp41 were found using the PC/GENE programs PALIGN and CLUSTAL to align available sequences of the T-cell surface glycoprotein CD4 (CD4 HUMAN) and the envelope polyprotein gp160 precursor (ENV-HV1-A2) using sequences from the protein sequence database SWISS-PROT, release 33. Once the octapeptide sequence SEQ ID NO:1: IVGGLVGL or its structural equivalent was identified as being common to the CD4 and HV proteins, the program PESEARCH was used to identify all other sequences in the database containing this sequence. Both the gp160 and the CD4 sequences were also used with the program FSTPSCAN to identify all related sequences. From the consensus sequence shown in Table 5, PESEARCH was used to identify all sequences containing related sequences. Subsequently BLAST2 searches using the Pasteur Institute (Paris) resource were run to update the data base of gp160 and CD4 sequences.

(2) Prediction of Transmembrane Helices

The method by Rao and Argos was used to predict sequences for transmembrane helices. Rao & Argos, European J Biochemistry 128: 565-575, 1982 was used to show predicted sequences from different species. These sequences are shown in Table 8.

(3) Construction of Models of Transmembrane Helices

In order to visualize the structures of the CD4 and HIV-1 octapeptide regions and to assess the structural effects of various replacements of the conserved glycine residues in the octapeptide in HIV-2 and SIV gp41 molecules, models were constructed (For example see conserved sequences of octapeptides in Table 8.

TABLE 8
[NEEDS TO BE CITED IN TEXT, PERHAPS IN PARA 268.]
Prediction of Transmembrane Helices Using the Method of Rao & Argos

| | Sequence | | | | | | | | | | | | | | | | | | | | |
|---|---|---|---|---|---|---|---|---|---|---|---|---|---|---|---|---|---|---|---|---|---|
| | Position | | | | | | | | | | | | | | | | | | | | |
| Species | Number | 1 | 2 | 3 | 4 | 5 | 6 | 7 | 8 | 9 | 10 | 11 | 12 | 13 | 14 | 15 | 16 | 17 | 18 | 19 | 20 |
| CD4 | | Q | P | M | A | L | I | V | G | G | V | A | G | L | L | L | F | I | G | L | G |
| HIV1- | | I | K | I | F | M | I | V | G | G | L | V | G | L | R | I | V | F | A | V | L |
| gp41 | | | | | | | | | | | | | | | | | | | | | |
| HIV2- | | Q | Y | G | V | H | I | V | V | G | I | I | A | L | R | I | A | I | Y | V | V |
| gp41 | | | | | | | | | | | | | | | | | | | | | |
| SIV- | | K | I | F | L | M | A | V | G | G | I | I | G | L | R | I | I | M | T | V | F |
| CZ | | | | | | | | | | | | | | | | | | | | | |
| Gp41 | | | | | | | | | | | | | | | | | | | | | |

This was done using SYBYL software running on a Silicon Graphics Indigo, for the transmembrane helix region in general and the octapeptide in detail for CD4, gp41 from HIV-1, gp41 from SIV-CZ, and gp41 from various HIV-2 species. All structures shown were constructed as helices and then subjected to global energy minimization, using standard computer protocols.

(4) Docking of Transmembrane Helix and Octapeptide Models for CD4 and HIV-1

To examine the possibility that the octapeptide sites of CD4 and gp41 interact directly, the transmembrane peptides of CD4 and gp41 of HIV-1 were manipulated using SYBYL to bring them into close proximity, taking into account both the helix dipole interactions and steric interactions.

b) Results

Initially, amino acid sequences were available from the gp41 of 26 HIV-1 isolates, representing wide temporal and geographic sources. The interstrain variation of some regions is great, while other regions are more conserved. Table 5 shows the alignment of octapeptide sequences from the gp41 of 26 HIV-1 isolates, representing wide temporal and geographic sources.

TABLE 5
Comparison of gp41 Sequences
HIV-1 Type

| HIV1 | Motif Residue Number | | | | | | | | |
|---|---|---|---|---|---|---|---|---|---|
| Type | 1 | 2 | 3 | 4 | 5 | 6 | 7 | 8 | 9 |
| HIV10 | I | V | G | G | L | V | G | L | R |
| HIV14 | I | V | G | G | L | V | G | L | R |
| IHV16 | I | V | G | G | L | I | G | L | R |
| HIV18 | I | V | G | G | L | I | G | L | R |
| HIV1A | I | V | G | G | L | V | G | L | R |
| HIV1J | I | V | G | G | L | V | G | L | R |
| HIV1F | I | V | G | G | L | I | G | L | R |
| HIV1S | I | V | G | G | L | V | G | L | R |
| HIV1G | I | V | G | G | L | V | G | L | R |
| HIV1O | I | V | G | G | L | V | G | L | R |
| HIV1L | I | V | G | G | L | V | G | L | R |
| HIV1R | I | V | G | G | L | V | G | L | K |
| HIV1P | I | V | G | G | L | V | G | L | R |
| HIV1V | I | V | G | G | L | V | G | L | R |
| HIV1M | V | V | G | G | L | I | G | L | R |
| HIV1E | I | I | G | G | L | I | G | L | R |
| HIV1Y | I | V | G | G | L | V | G | L | R |
| HIV1B | I | V | G | G | L | V | G | L | R |
| HIV1X | I | V | G | G | L | V | G | L | R |
| HIV1D | I | V | G | G | L | I | G | L | R |
| HIV1C | I | V | G | G | L | I | G | L | R |
| HIV1W | I | V | G | G | L | I | G | L | R |
| HIV1Z | I | V | G | G | L | I | G | L | R |
| HIV1L | I | V | G | G | L | I | G | L | R |
| HIV1H | I | V | G | G | L | I | G | L | R |
| HIV1K | I | V | G | G | L | I | G | L | R |
| Consen- | I(25) | V(25) | G(26) | G(26) | L(26) | V(14) | G(26) | L(26) | R(25) |
| sus | | | | | | | | | |
| | V(1) | I(1) | | | | I(12) | | | K(1) |
| SIV-CZ | A | V | G | G | I | I | G | L | R |

shows sequences of these 26 strains of HIV-1 beginning at approximate residue 688 of gp160. Positions 1, 2 and 6 contain the functionally conserved hydrophobic residues, isoleucine (I) and valine (V), with isoleucine dominating at position 1, valine dominating at position 2, and neither dominating at position 6. Leucine (L) is conserved throughout positions 5 and 8. Glycine (G) is conserved throughout positions 3, 4 and 7. Position 9 predominantly contains arginine (R), which is substituted by another positively charged residue, lysine (K), in HIV-1 RH. Table 5 also shows the relationship of the sequence found in HIV-1 to that in the genetically related simian virus SIV-CZ. With the exception of positions 1 and 5, SIV-CZ does not differ from the HIV-1 consensus; however, these positions are conservatively replaced by other hydrophobic residues. An additional 664 HIV-1 isolates were examined, with similar results (not tabulated): glycine was always conserved at position 7 and no other amino acid other than alanine (next smallest to glycine) was found at positions 3 or 4 (not both) in 243 of the total 690 sequences examined.

Table 6, shows the corresponding sequences in strains of HIV-2 and genetically related SIV (with the SIV-CZ sequence and the consensus of the HIV-1 sequences for comparison and contrast). Position 1 contains hydrophobic residues throughout HIV-2; however, SIV-AG has aspartic acid (D), a negatively charged residue, at position 1.

TABLE 6
Comparison of gp41 Sequences

| | Motif Residue Number | | | | | | | | |
|---|---|---|---|---|---|---|---|---|---|
| | 1 | 2 | 3 | 4 | 5 | 6 | 7 | 8 | 9 |
| HIV2 | | | | | | | | | |
| Type | | | | | | | | | |
| HIV2R | I | I | V | A | V | I | A | L | R |
| HIV2C | I | V | V | G | I | I | V | L | R |
| IHV2L | I | V | V | G | I | I | G | L | R |
| HIV2G | I | V | V | G | V | I | V | L | R |
| HIV2N | V | V | V | G | I | V | A | L | R |
| HIV2S | I | V | V | G | I | I | V | L | R |
| HIV2I | I | V | V | G | I | V | A | L | R |
| HIV2B | I | V | V | G | I | I | A | L | R |
| SIV | | | | | | | | | |
| Type | | | | | | | | | |
| SIV- | V | V | V | G | V | I | L | L | R |
| ML | | | | | | | | | |
| SIV- | V | V | V | G | V | I | L | L | R |
| MK | | | | | | | | | |
| SIV-AT | V | I | V | G | I | I | G | L | R |
| SIV-1A | A | V | I | G | V | I | G | L | R |
| SIV-AG | D | V | L | G | I | I | G | L | R |
| SIV-GB | L | V | L | G | I | I | G | L | R |
| SIV-SP | I | V | L | G | V | I | G | L | R |
| SIV-M1 | I | I | V | G | V | I | L | L | R |
| SIV-S4 | I | V | L | G | V | I | G | L | R |
| HIV1 | I(25) | V(25) | G(26) | G(26) | L(26) | V(14) | G(26) | L(26) | R(25) |
| Consen- | | | | | | | | | |
| sus | | | | | | | | | |
| | V(1) | I(1) | | | | I(12) | | | K(1) |
| SIV-CZ | A | V | G | G | I | I | G | L | R |

Positions 2, 5 and 6 contain functionally conserved hydrophobic residues, with valine dominating at position 2, isoleucine and valine sharing position 5, and isoleucine dominating position 6. Unlike HIV-1 and SIV-CZ, however, positions 3, 4 and 7 of HIV-2 do not have completely conserved glycines. Only in position 4 of SIV is glycine conserved. Hydrophobic residues are always present in position 3. Position 4 of HIV2-RO contains an alanine instead of glycine, and position 4 of SIV A1 contains isoleucine instead of glycine. Position 7 contains an array of glycines, alanines, valines, and leucines. Positions 8 and 9 have completely conserved leucine and arginine residues, respectively. An additional 9 HIV-2 strains were examined (not tabulated) and consistently lacked glycine at positions 3 and 7. No HIV2 sequences containing a single alanine residue in the three conserved positions, 3, 4 and 7, were observed, with the majority substituting the bulky valine in position 3 of this motif.

Table 7 shows sequences in the TM domain in the CD4 protein of humans and several other species of interest.

TABLE 7
Comparison of CD4 Sequences

| | Residue Number | | | | | | | |
|---|---|---|---|---|---|---|---|---|
| Species | 1 | 2 | 3 | 4 | 5 | 6 | 7 | 8 |
| Human | V | L | G | G | V | A | G | L |
| Macaque | V | L | G | G | V | A | G | L |
| Mouse | V | L | G | G | S | F | G | F |
| Chimpanzee | V | L | G | G | V | A | G | L |
| Rat | V | L | G | S | A | F | S | F |
| Cat | V | L | G | G | V | L | G | L |
| Rabbit | A | L | G | G | T | A | G | L |
| Whale | V | L | G | G | I | T | S | L |

Valine is completely conserved at position 1, and leucine at position 2. Similar to HIV-1 and SIV-CZ, glycines are conserved at positions 3, 4 and 7, with the exception of Rat CD4, which has serine substituted at positions 4 and 7. Position 5 shows conserved hydrophobic residues, except in mouse CD4, which has serine. Positions 6 and 8 show hydrophobic residues throughout. Thus positions 1-8 of CD4 of humans and at least two other primates resemble the highly conserved octapeptide sequence in positions 1-8 of the gp41 of HIV-1 and SIV-CZ (although not the conserved, positively charged residue in position 9). (Table 7) also shows the TM sequences of the Fusin co-receptor and a potential HIV receptor from the human brain (the possible Opioid Receptor, OPRY-HUMAN). Note the same sequence is in both CCR5 and CXCR4. The Fusin receptor has three glycine residues spaced similarly to the CD4 TM region, but inverted in order, while the putative brain receptor has the conserved glycine residues in the same order as CD4. Thus, known or putative receptors for HIV have a structurally similar sequence as discovered to exist in the CD4 TM region.

Since the existence of the “notch” in the helix (described herein) depends on this helical structure, the structure of the conserved TM region was experimentally determined, embedded in a detergent micelle to mimic the hydrophobic interior of the lipid membrane. The octapeptide corresponding to these conserved residues in CD4 was chemically synthesized using standard fmoc technology and purified by reverse-phase high-pressure liquid chromatography. The peptide was then incorporated into a deuterated detergent micelle and its three-dimensional structure determined by proton nuclear magnetic resonance specroscopy (NMR) at 600 MHz. The NH region of the proton NMR NOESY spectrum showed i to i+3 and i to i+4 cross peaks demonstrating the alpha helical structure of this region of the TM peptide.

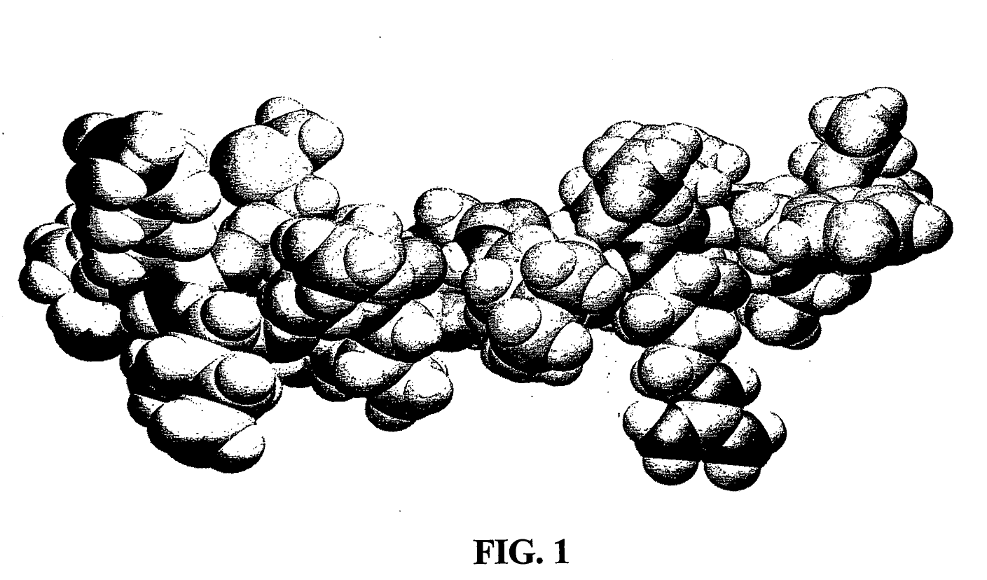

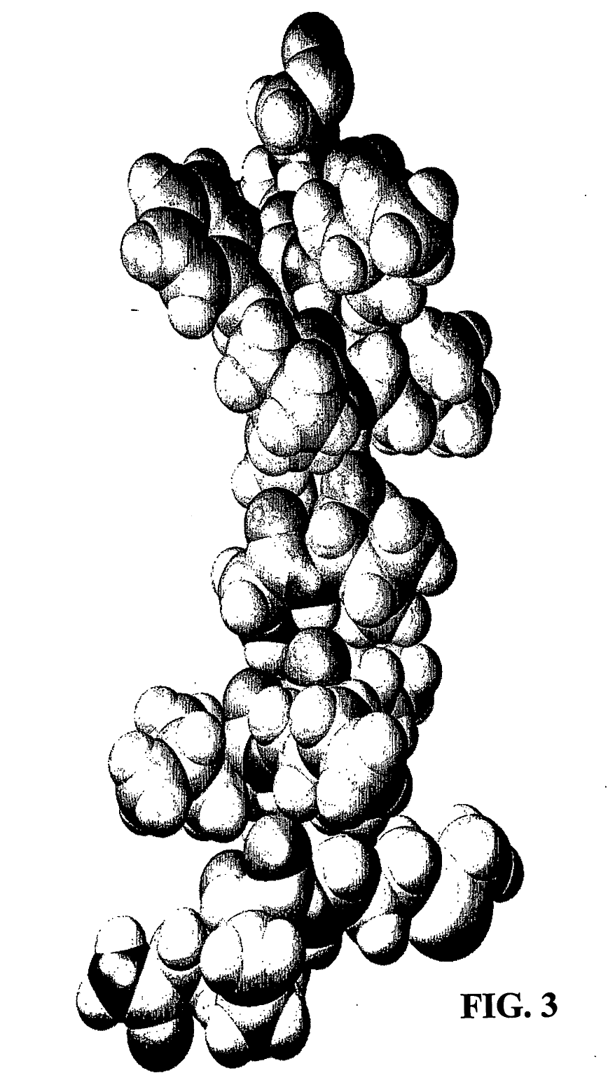

FIGS. 1, 2, and 3 show computer-generated models of the Van der Waals surfaces of the transmembrane sequences of representative strains of HIV-1 and HIV-2, and of human CD4 respectively. A glycine surface resembling a “notch” can be seen in the helices of both HIV-1 (FIG. 1) and of CD4 (FIG. 3). A similar notch would be generated by the corresponding sequences of fusins and OPRY-HUMAN (not shown).

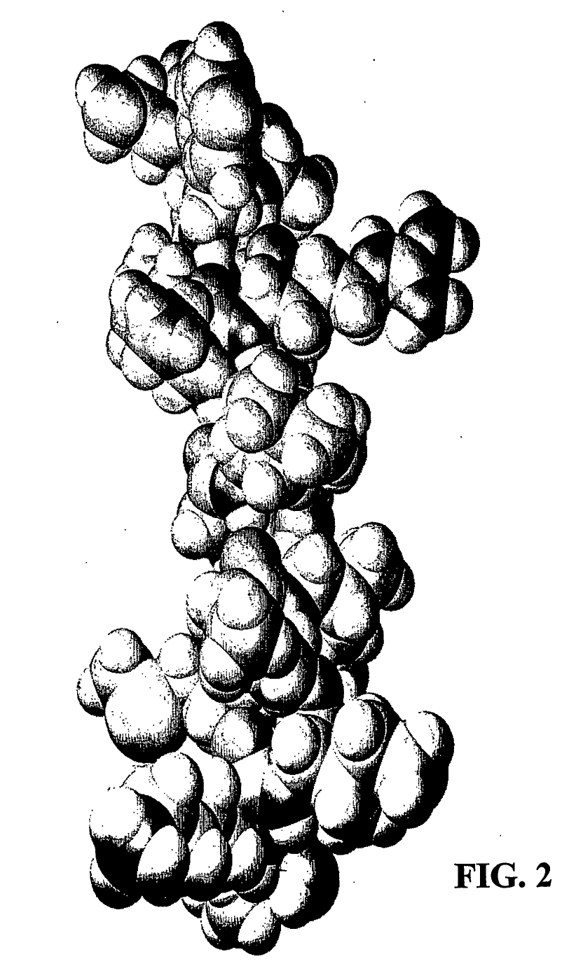

As shown in FIG. 2, the notch is absent in HIV-2 strain HV2D1, due to a single protruding valine side chain. (Kuhnel, H., et al., Nucleic Acids Res. 18 (20), 6142 (1990)). The minimum perturbation in other HIV2 sequences is at least one alanine and one valine. HV2D1 is the least perturbed of the notch sequences, having valine instead of glycine only in position 3. HV2S2 lacks glycines in positions 3 and 7, and HV2RO lacks glycines in positions 3, 4 and 7; as would be expected, modeling shows the notch site in these strains to be occluded also (not shown). Thus the notch disappears when one, two, or three glycines are substituted with hydrophobic residues larger than alanine [-note alanine can also inhabit position 1 or 3.

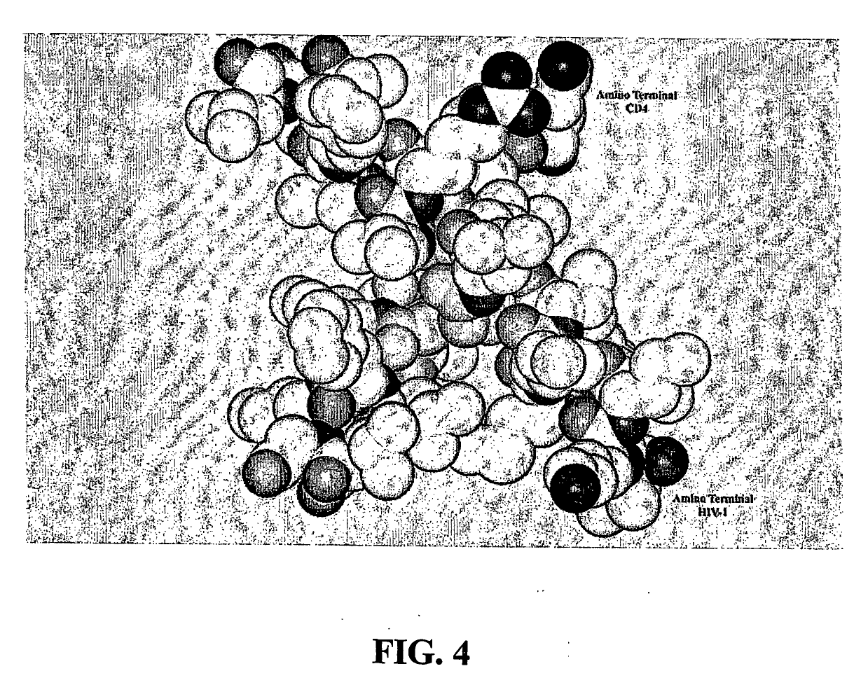

The notch sequences of HIV-1 gp 160 and CD4 can bind directly to each other through the notch sites. Thus, FIG. 4 shows the HIV-1 and CD4 octapeptides docked, with the grooves oriented opposite each other in a cross-shaped configuration. This orientation maximizes both helix dipole interactions and steric interactions. A similar attempt to show docking to CD4 was made with the minimally perturbed HIV-2 strain HV2131: the absence of glycine at position 3 (which contains a valine) disrupts docking of the two helices. The membrane is not thought to prevent the ability to make an x-like orientation when the disclosed compositions are in the membrane as the structure results from helix dipole interactions superimposed on a notch fit which will be maximized in the membrane.

CD4 and the above-mentioned known and putative co-receptor molecules of the host have structurally similar octapeptide sites. In the process of evolving to high virulence for humans, HIV-1 may have mimicked these sites. The CD4 octapeptide was shown by two-dimensional NMR techniques conducted in a membranous environment to assume an alpha-helical structure. Thus this and the structurally related octapeptide sequences, based on computer modeling, would have a notch structure within membranes, consistent with the region having a discrete functional domain. The computer modeling disclosed herein shows that the HIV-1 and host notch sites can interact functionally with each other, and would be able to functionally bind a common ligand similarly. Both HIV-1 and HIV-2 (which lacks the notch) have arginine (or occasionally lysine) in position 9.

### 2. Example 2—Antiviral Assays.

Candidate molecules with empirical or hypothetical capacity to bind to the target or its ligand can be further tested for antiviral activity and (lack of) cytotoxicity in cell culture systems in vitro. For example, production of the viral protein P24 in human peripheral blood mononuclear cells (PBMC) exposed to cell-free virus of a clinical isolate of HIV-1 reflects the capacity of the virus to progress through the complete replication cycle, and the quantity of P24 is readily detected in culture by immunologic assay as described by Jiang et al, Journal of Experimental Medicine 174:1557, 1991. Because mere cytotoxic activity of the candidate would diminish P24 production (in the absence of specific antiviral effect), the cells would be examined for microscopic indications of toxicity and for capacity to exclude a vital dye, such as MTT.

Antiviral effects (IC90) should exceed cytotoxic effects (IC30) by about 100-fold if a compound is to be considered for further testing in vivo. Candidates, for example, molecules identified through molecular modeling as binding the notch sequence with energy minimizations ranging from less than 4, or 3, or 2, or 1 Angstroms can be tested in P24 assays with strains representing the known subtypes A-F of HIV-1. Also disclosed are molecules that have a range of afinities that bind to the “notch: sequence or its target, with dissociation constants from 10⁻³ M to 10⁻¹⁵ M, with each amount in between this range also disclosed.

A candidate molecule can less readily inhibit the overall replication cycle and more readily inhibit the above-mentioned fusion process. Thus candidates can also be tested for capacity to inhibit HIV-1-mediated cell fusion in vitro; virus-infected cells of a cultivable line such as H-9 can be labeled with the fluorescent dye BCECF-AM, mixed and incubated with an excess of uninfected cells, and labeled aggregates can be scored by fluoromicroscopy as described by Jiang et al, Biochemical and Biophysical Research Communications 195:533, 1993. Alternatively,the formation of syncytia can be scored by simple microscopy. The fusion assay and other in vitro procedures will be used to determine which of the known steps of the replication cycle is inhibited by a candidate molecule. For example, in the absence of an effect in the fusion assay, the inhibition of nuclear uptake of viral RNA from “pseudovirions”, as described by Thomas et al, Viral Immunology 9:73, 1996, would indicate interference with a post-fusion process prior to reverse transcription of the viral RNA in the cell nucleus. Localizing the mechanism of antiviral action of a candidate molecule would be useful in suggesting which category of known anti-HIV drugs might be synergistic with the candidate. Candidate molecules with a high ratio of antiviral/cytotoxic activity in vitro are predictive of molecules having activity in vivo. In vivo analysis can be performed with SCID mice: due to the host-range restriction of HIV, readily available laboratory animal species are not suitable; however, mice with “severe combined immunodeficiency” (SCID) can be reconstituted with human immune system cells, and these hybrids can be used for initial in vivo testing of promising candidate molecules-before testing in chimpanzees or humans.

### 3. Example 3

The NH “helix” signature region of a 600 MHz NMR Spectrum of a peptide designed based on the HIV1 “notch” sequence embedded in SDS Micelles to mimic the membrane environment has been performed. These experiments directly demonstrated that the peptide region encompassing the glycine surfaced “notch” described here is in fact helical when in a hydrophobic environment such as would be found in a cell membrane (here mimicked by an SDS micelle). This region is has been represented graphically through molecular modeling as described herein for the appropriate HIV regions in both HIV1 and HIV2 types, demonstrating that the “notch” will be blocked in all HIV2 variants but present in all HV1 variants described to date. These modeling events show that even a single Valine substitution found in some HIV2 variants blocks the “notch” region. Modeling has also been performed between the CD4 notch and the HIV-1 notch and these results show that an interaction between this notch region of HIV1 and a conserved notch region found in the cell surface receptor CD4 can take place. An example of a molecular model of an HIV-1 notch and a CD4 notch can be seen in FIG. 4.

F. Sequences

| SEQ ID NO:1: | IVGGLVGL Viral notch |
|---|---|
| SEQ ID NO:2: | VLGGVAGL CD4 notch |
| SEQ ID NO:3: | IGYFGGIF |
| SEQ ID NO:4: | CVGGLLGN |
| SEQ ID NO:5: | IVGGVAGLLL |
| SEQ ID NO:6: | IVGGLVGLR |
| SEQ ID NO:7: | EGGVLGGVAGLLL, |
| SEQ ID NO:8: | QPMALIVGGVAGLLLFIGLGIFFCVR |
| SEQ ID NO:9: | MIVGGLVGLR |
| SEQ ID NO:10: | YIKIFMIVGGLVGLRIVFAVLSIVNR |
| SEQ ID NO:11: | GAVIGIGALFLGFLGAAGSTMGAASMTLTVGAR |
| SEQ ID NO:12: | GFLAAGSTMG |
| SEQ ID NO:13: | XXGGXXGX where X is any amino acid other than glycine |
| SEQ ID NO:14: | XXAGXXGX where X is any amino acid other glycine |
| SEQ ID NO:15: | XXGAXXGX where X is any amino acid other than glycine |
| SEQ ID NO:16: | I/V V/I GGX I/V GX |
| SEQ ID NO:17: | I/V V/I AGX I/V GX |
| SEQ ID NO:18: | I/V V/I GAX I/V GX |
| SEQ ID NO:19: | I/V V/I GGL I/V GL |
| SEQ ID NO:20: | I/V V/I AGL I/V GL |
| SEQ ID NO:21: | I/V V/I GAL I/V GL |
| SEQ ID NO:22: | XXGGXXGX, wherein X is (any amino acid with a hydrophobic |
| | sidechain). |
| SEQ ID NO:23: | XXAGXXGX, wherein X is (any amino acid with a hydrophobic |
| | sidechain). |
| SEQ ID NO:24: | XXGAXXGX, wherein X is (any amino acid with a hydrophobic |
| | sidechain). |
| SEQ ID NO 25 | Z(X)n)VLGGVAGLLL |
| SEQ ID NO 26: Accession No. CAD59666 GP160 complete protein sequence | |
| 1 mrakgirniy qrlwrwgmml lgmlmicsat eklwvtvyyg vpvwkeaitt lfcasdakay | |
| 61 dtevbnvwat hacvptdpnp qevilenvte nfnmgknnmv eqmhediisl wdqslkpcvk | |
| 121 ltplcvtlnc tglkknatnt tssnkgamee gemlmcsfnv ttsigdrmqr eyalfykldi | |
| 181 vpvdgdnstr yrliscntsv itqacpkvsf epipihycap agfailkcnn kkfngtgpct | |
| 241 nvstvqcthg irpvvstqll lngslaeeev virstnlsdn aktiivqlkd pveikctrpn | |
| 301 nntrksipig pgrafyatgd iigdirqahc nlsstnwtna lkqigkelrk qfknktiifn | |
| 361 qssggdpeiv mhsfncggef fycdstqlih ntwngtewpd ddititlpcr ikqiimnwqe | |
| 421 vgkamyappi rgriecssni tgllltrdgg inntngsetf rpgggdmrdn wrselykykv | |
| 481 vkieplgvap tkakrrvvqr ekraalgavf lgflgaagst mgaasmtltv qarlllsgiv | |
| 541 qqqnnllrai eaqqhllqlt vwgikqlqar vlavekylkd qqllgiwgcs gklictttvp | |
| 601 wnaswsnksl seiwdnmtwm ewereinnyt sliysliees qnqqekneqe lleldkwasl | |
| 661 wnwfnitqwl wyikifimiv gglvglrivf avlsivnrvr qgysplsfqt hlpiprgpdr | |
| 721 pegieeegge rdrdrsirlv ngslaliwdd lrslclfsyh rlrdlllivt rivellgrrg | |
| 781 wealkyrwnl lqywsqelkn savnllnata iavaegtdrv ievlqaayra irhiprrirq | |
| 841 glerill | |
| SEQ ID NO:27 Accession AJ535619 GP160 complete cDNA sequence | |
| 1 atgagagcga aggggatcag gaggaattat cagcgcttgt ggagatgggg catgatgctc | |
| 61 cttgggatgt tgatgatctg tagtgctaca gaaaaattgt gggtcacagt ctattatggg | |
| 121 gtacctgtgt ggaaagaagc catcaccact ctattttgtg catcagatgc taaagcatat | |
| 181 gatacagagg tacataatgt ttgggccaca catgcctgtg tacccacaga ccccaaccca | |
| 241 caagaagtaa tattggaaaa tgtgacagaa aattttaaca tggggaaaaa taacatggta | |
| 301 gaacagatgc atgaggatat aatcagttta tgggatcaaa gcctaaagcc atgcgtaaaa | |
| 361 ttaaccccac tctgtgttac tttaaattgc actggtctga agaagaatgc tactaatacc | |
| 421 actagtagta acaagggagc gatggaggaa ggagaaatga aaaactgctc tttcaatgtc | |
| 481 accacaagca taggagatag gatgcagaga gaatatgcac ttttttataa acttgatata | |
| 541 gtaccagtag atggtgataa tagtaccaga tataggttga taagttgcaa cacctcagtc | |
| 601 attacacagg cttgtccaaa ggtatccttt gagccaattc ccatacatta ttgtgccccg | |
| 661 gctggttttg cgattctaaa gtgtaacaat aagaagttca atggaacagg accatgtaca | |
| 721 aatgtcagca cagtacaatg tacacatgga attaggccag tagtatcgac tcaactgctg | |
| 781 ttaaatggca gtctagcaga agaagaggta gtaattagat ctaccaatct ctcggacaat | |
| 841 gctaaaacca taatagtaca gctaaaagac cctgtagaaa ttaagtgtac aagacccaac | |
| 901 aacaatacaa gaaaaagtat acctatagga ccagggagag cattttatgc aacaggagac | |
| 961 ataataggag atataagaca agcacattgt aaccttagtt caacaaactg gactaacgct | |
| 1021 ttaaaacaga taggtaaaga attaagaaaa cagtttaaga ataaaacaat aatctttaat | |
| 1081 caatcctcag gaggggaccc agaaattgta atgcacagct ttaattgtgg aggggaattt | |
| 1141 ttctactgtg attcaacaca actgtttaat aatacttgga atggtactga atggccagat | |
| 1201 gacgatataa ctatcacact cccatgcaga ataaaacaaa ttataaacat gtggcaggaa | |
| 1261 gtaggaaaag caatgtatgc ccctcccatc agaggacgaa ttgaatgttc atcaaatatt | |
| 1321 acaggactac tactaacaag agatggtggt attaataaca cgaatgggag cgagaccttc | |
| 1381 agacctggag gaggagatat gagggacaat tggagaagtg aattatataa atataaagta | |
| 1441 gtaaaaatag aaccattagg agtagcaccc accaaggcaa agagaagagt ggtgcagaga | |
| 1501 gaaaaaagag cagcattagg agctgtgttc cttgggttct taggagcagc aggaagcact | |
| 1561 atgggcgcag cgtcgatgac gctgacggta caggccagac tattgttgtc tggtatagtg | |
| 1621 caacagcaga acaatttgct gagggctatt gaggcgcaac agcatctgtt gcaactcaca | |
| 1681 gtctggggca tcaagcagct ccaggcaaga gtcctggctg tggaaaaata cctaaaggat | |
| 1741 caacagctcc tggggatttg gggttgctct ggaaaactca tttgcaccac tactgtgccc | |
| 1801 tggaatgcta gttggagtaa taaatctctg agtgagattt gggataacat gacctggatg | |
| 1861 gagtgggaaa gagaaattaa caattacaca agcttaatat acagcttaat tgaagaatcg | |
| 1921 caaaaccaac aagagaagaa tgaacaagaa ttattagaat tggataaatg ggcaagtctg | |
| 1981 tggaattggt ttaacataac acaatggctg tggtatataa aaatattcat aatgatagta | |
| 2041 ggaggcttgg taggtttaag aatagttttt gctgtactct ctatagtgaa tagagttagg | |
| 2101 cagggatatt caccattatc gtttcagacc cacctcccaa tcccgagggg acccgacagg | |
| 2161 cccgaaggaa tagaagaaga aggtggagag agagacagag acagatccat tcgattagtg | |
| 2221 aacggatcct tagcacttat ctgggacgat ctgcggagcc tgtgcctctt cagctaccac | |
| 2281 cgcttgagag acttactctt gattgtaacg aggattgtgg aacttctggg acgcaggggg | |
| 2341 tgggaagccc tcaaatatcg gtggaatctc ctacagtatt ggagtcagga actaaagaat | |
| 2401 agtgctgtta acttgctcaa tgccacagcc atagcagtag ctgaggggac agatagggtt | |
| 2461 atagaagtat tacaagcagc ttatagagct attcgccaca tacctagaag aataagacag | |
| 2521 ggcttggaaa ggattttgct ataa | |
| SEQ ID NO:28: | EGG(VL)GG(VA)GLLL (Related to SEQ ID NO:1) |
| (SEQ ID NO: | 676-702 plus KKKC, (TNWLWYIKLFIMIVGGLVGLRIVFAKKKC) |
| 29) | |
| SEQ ID NO:30 | QPMALIVGGLVGLLLFIGLGIFFCVR (Related to SEQ ID NO:1) |
| SEQ ID NO:31 | HIGFGGIF |
| SEQ ID NO:32: | VGGLLGNC |
| SEQ ID NO:33: | IVGGLVGLLL, derived exactly from 1] |
| SEQ ID NO:34 | EGGIVGGVAGLLL[G]_{X}[R]_{y} (SEQ ID NO 34), [G]ₓ is a flexible |
| | glycyl linker of any length such as 1, 2, 3, 4, 5, 6, 7, |
| | 8, or 9 [R]_{y} are argimines, any length, such as 1, 2, 3, |
| | 4, 5, 6, 7, 8, or 9. |
| SEQ ID NO:35 | FMIVGGLVGLRIV |
| SEQ ID NO:36: | ALVLGGVAGLLLF |

What is claimed is:

1. A composition for reducing HIV infectivity comprising a molecule that binds the 5 notch structure formed by the amino acids set forth in SEQ ID NO:1.

2. (canceled)

3. (canceled)

4. (canceled)

5. (canceled)

6. (canceled)

7. A method for reducing interactions between CD4 and HIV gp160, comprising incubating an inhibitor of the interaction between CD4 and gp160 with CD4 and gp160, and wherein the inhibitor can interact with a domain having a structure homologous to the structure produced by the amino acids set forth in SEQ ID NO: 1, and wherein the inhibitor has an activity in a p24 assay.

8. A method for inhibiting HIV infectivity comprising administering an inhibitor of the interaction between CD4 and HIV gp160, wherein the inhibitor can interact with amino acids of SEQ ID NO:1, and wherein the inhibitor has an activity in a p24 assay.

9. A method of treating a subject comprising administering to the subject an inhibitor of HIV infectivity, wherein the inhibitor reduces the interaction between CD4 and HIV gp160, and wherein the subject is in need of such treatment, wherein the inhibitor can interact with amino acids of SEQ ID NO:1, and wherein the inhibitor has an activity in a p24 assay.

10. (canceled)

11. (canceled)

12. (canceled)

13. (canceled)

14. (canceled)

15. A method of identifying an inhibitor of an interaction between CD4 and gp160 comprising incubating a set of molecules with a CD4 notch domain-gp160 notch domain complex, and isolating the molecules that can disrupt the interaction between CD4 notch domain and the gp160 notch domain, wherein the interaction disrupted comprises an interaction between the CD4 notch domain and an amino acid of the gp160 notch domain.

16. The method of claim 15, wherein the CD4 notch domain-gp160 notch domain complex comprises an energy transfer pair, wherein the energy transfer pair comprise an energy donor and an energy acceptor.

17. The method of claim 16, wherein the step of isolating further comprises assaying fluorescence of the energy transfer pair.

18. The method of claim 17, wherein the step of isolating further comprises selecting a molecule that inhibits the fluorescence.

19. The method of claim 17, wherein the energy transfer pair comprises a donor molecule that emits fluorescence whose wavelength overlaps that of the absorption band of an acceptor molecule, resulting in quenching of the donor molecule fluorescence and/or sensitization of acceptor molecule fluorescence.

20. (canceled)

21. (canceled)

22. (canceled)

23. A composition identified by the process of claim 15.

24. A composition capable of being identified by the process of claim 15.

25. A method of manufacturing a composition for inhibiting the interaction between CD4 and gp160 comprising synthesizing the inhibitor of claim 15.

26. (canceled)

27. A method of manufacturing a composition for inhibiting the interaction between CD4 and gp160 comprising admixing the inhibitor with a pharmaceutical carrier.

28. (canceled)

29. (canceled)

30. (canceled)

31. (canceled)

32. A method of malting a composition capable of inhibiting HIV infectivity comprising admixing a compound with a pharmaceutically acceptable carrier, wherein the compound is identified by administering the compound to a system, wherein the system supports HIV infectivity via a CD4 notch-gp160 notch interaction, assaying the effect of the compound on the amount of HIV infectivity in the system, and selecting a compound which causes a decrease in the amount of HIV infectivity in the system because of an inhibition of the CD4 notch-gp160 notch interaction, relative to the system without the addition of the compound.

33. (canceled)

34. (canceled)

35. (canceled)

36. (canceled)

37. (canceled)

38. A method for reducing interactions between CD4 and HIV gp160, comprising incubating an inhibitor of the interaction between CD4 and g160 with CD4 and gp160, wherein the inhibitor can interact with at least one atom selected from the group consisting of the group of atoms set forth in Tables 3 and 4, and wherein the inhibitor has an activity in a p24 assay.

39. (canceled)

40. (canceled)

41. (canceled)

42. (canceled)

43. A method for reducing HIV infectivity, comprising incubating an inhibitor of the interaction between a gp160 notch molecule and a partner, wherein the inhibitor can interact with at least one atom selected from the group consisting of the group of atoms set forth in Tables 3 and 4, and wherein the inhibitor has an activity in a p24 assay.

44. (canceled)

45. (canceled)

46. (canceled)

47. (canceled)

48. (canceled)

49. A method of characterizing protein structures comprising the steps: (a) determining a gp160 notch domain three-dimensional structure; (b) determining an experimental protein three-dimensional structure; (c) comparing the experimental protein three-dimensional structure to the gp160 notch domain three-dimensional structure; and (d) recording variances between the gp160 notch domain three-dimensional structure and the experimental protein three-dimensional structure.

50. (canceled)

51. (canceled)

52. A method of evaluating two or more experimental proteins with respect to the gp160 notch domain, comprising: (i) evaluating the variances of (d) of claim 5 for a first experimental protein; (ii) evaluating the variances of (d) of claim 5 for a second experimental protein; and (iii) ranking the experimental protein with the least variance from the structure of gp160 notch domain as being most similar.

53. A method of displaying a representation of a gp160 notch domain comprising:
determining the three-dimensional coordinates of atoms of a gp160 notch domain;
providing a computer having a memory means, a data input means, a visual display means, the memory means containing three-dimensional molecular simulation software operable to retrieve coordinate data from the memory means and to display a three-dimensional representation of a molecule on the visual display means and being operable to produce a representation of an analog of the molecule responsive to operator-selected changes to the chemical structure of the molecule and to display the representation of the analog;
inputting three-dimensional coordinate data of the atoms of the gp160 notch domain into the computer and storing the data in the memory means;
displaying the representation of the gp160 notch domain on the visual display means.

54. A method of displaying a representation of an analog of a gp160 notch domain comprising:
a) determining the three-dimensional coordinates of atoms of a gp160 notch domain;
b) providing a computer having a memory means, a data input means, a visual display means, the memory means containing three-dimensional molecular simulation software operable to retrieve coordinate data from the memory means and to display a three-dimensional representation of a molecule on the visual display means and being operable to produce a representation of an analog of the molecule responsive to operator-selected changes to the chemical structure of the molecule and to display the representation of the analog;
c) inputting three-dimensional coordinate data of the atoms of the gp160 notch domain into the computer and storing the data in the memory means;
d) displaying the representation of the gp160 notch domain on the visual display means;
e) inputting into the data input means of the computer at least one operator-selected change in chemical structure of the gp160 notch domain forming a gp160 notch domain analog structure;
f) executing the molecular simulation software to produce a modified three-dimensional molecular representation of the analog structure; and
g) displaying the representation of the analog structure on the visual display means, whereby changes in three-dimensional structure of the gp160 notch domain consequent on changes in chemical structure can be visually determined.

55. (canceled)

56. (canceled)

57. A method for identifying the gp160 notch domain analogs comprising: producing a multiplicity of analog structures of the gp160 notch domain by the method of claim 11, and selecting an analog structure with a structure of the notch binding domain which is substantially like the gp160 notch domain.

58. (canceled)

59. A method for identifying a potential ligand of a protein comprising a gp160 notch domain comprising:
a) using a three-dimensional structure of the gp160 notch domain function or portions thereof formed from the atomic coordinates of the gp160 notch domain;
b) employing the three-dimensional structure to design or select the potential ligand.

60. (canceled)

61. (canceled)

62. (canceled)

63. (canceled)

64. (canceled)

65. (canceled)

66. (canceled)

67. A ligand of a gp160 notch domain containing polypeptide made according claim 52.

68. An apparatus for determining whether a compound will interact with a protein containing a gp160 notch domain, comprising:
a) a memory that stores a set of coordinates and identities of the atoms of the gp160 notch domain that together form a solvent-accessible surface; and executable instructions; and
b) a processor, wherein the executes instructions to receive structural information for a candidate compound; determine if the structure of the candidate compound is complementary to the structure of the solvent-accessible surface of the gp160 notch domain; and output the results of the determination.

69. (canceled)

70. (canceled)

71. (canceled)

72. A computer-readable storage medium comprising digitally-encoded structural data, wherein the data comprise the identity and three-dimensional coordinates, or coordinates providing a structural homolog, of at least 2 amino acids set forth in SEQ ID NO:1.

73. (canceled)

74. (canceled)

75. (canceled)

76. An apparatus comprising computer-readable storage medium and software wherein the apparatus can
a) receive a subject set of coordinates for a subject structure;
b) compare the subject set of coordinates to a reference set of coordinates related to the gp160 notch domain;
c) calculate the root mean squared deviation of the subject set of coordinates from the reference set of coordinates; and
d) compare the root mean squared deviation to limit values, whereby if the root mean square deviation is less than or equal to the limit values, the subject structure is assigned a function based on the subject structure's similarity to the reference structures.

77. (canceled)

78. (canceled)

79. A method of determining relationships between two or more polypeptide structures, comprising:
a) obtaining a reference structure, wherein the reference structure is a structure of a polypeptide comprising the gp160 notch domain or a portion thereof;
b) obtaining at least one subject structure;
c) determining a reference structure topology diagram and a subject structure topology diagram;
d) comparing the reference structure topology diagram and the subject structure topology diagram; and
e) assigning a relationship between the reference structure and any subject structure based on deviations between the reference structure and subject structure.

80. (canceled)

81. (canceled)

82. (canceled)

83. (canceled)

84. (canceled)

85. (canceled)

86. A method of identifying an inhibitor of an interaction with a CD4 notch comprising incubating a set of molecules with a CD4 notch domain, and isolating the molecules that bind the CD4-notch.

87. (canceled)

88. A method of identifying an inhibitor of an interaction with a gp160 notch comprising incubating a set of molecules with a gp160 notch domain, and isolating the molecules that bind the gp160-notch.

89. (canceled)


SEQUENCE LISTING (36 sequences)

SEQ ID NO: 1 - protein, 8 aa, Artificial Sequence
  Description of Artificial Sequence; Note = Synthetic  Construct
IVGGLVGL

SEQ ID NO: 2 - protein, 8 aa, Artificial Sequence
  Description of Artificial Sequence; Note = Synthetic  Construct
VLGGVAGL

SEQ ID NO: 3 - protein, 8 aa, Artificial Sequence
  Description of Artificial Sequence; Note = Synthetic  Construct
IGYFGGIF

SEQ ID NO: 4 - protein, 8 aa, Artificial Sequence
  Description of Artificial Sequence; Note = Synthetic  Construct
CVGGLLGN

SEQ ID NO: 5 - protein, 10 aa, Artificial Sequence
  Description of Artificial Sequence; Note = Synthetic  Construct
IVGGVAGLLL

SEQ ID NO: 6 - protein, 9 aa, Artificial Sequence
  Description of Artificial Sequence; Note = Synthetic  Construct
IVGGLVGLR

SEQ ID NO: 7 - protein, 13 aa, Artificial Sequence
  Description of Artificial Sequence; Note = Synthetic  Construct
EGGVLGGVAGLLL

SEQ ID NO: 8 - protein, 26 aa, Artificial Sequence
  Description of Artificial Sequence; Note = Synthetic  Construct
QPMALIVGGVAGLLLFIGLGIFFCVR

SEQ ID NO: 9 - protein, 10 aa, Artificial Sequence
  Description of Artificial Sequence; Note = Synthetic  Construct
MIVGGLVGLR

SEQ ID NO: 10 - protein, 26 aa, Artificial Sequence
  Description of Artificial Sequence; Note = Synthetic  Construct
YIKIFMIVGGLVGLRIVFAVLSIVNR

SEQ ID NO: 11 - protein, 33 aa, Artificial Sequence
  Description of Artificial Sequence; Note = Synthetic  Construct
GAVIGIGALFLGFLGAAGSTMGAASMTLTVGAR

SEQ ID NO: 12 - protein, 10 aa, Artificial Sequence
  Description of Artificial Sequence; Note = Synthetic  Construct
GFLAAGSTMG

SEQ ID NO: 13 - protein, 8 aa, Artificial Sequence
  Description of Artificial Sequence; Note = Synthetic  Construct
  VARIANT 1, 2, 5, 6, 8,: Xaa = Any Amino Acid other than Glycine
XXGGXXGX

SEQ ID NO: 14 - protein, 8 aa, Artificial Sequence
  Description of Artificial Sequence; Note = Synthetic  Construct
  VARIANT 1, 2, 5, 6, 8: Xaa = Any Amino Acid other than Glycine
XXAGXXGX

SEQ ID NO: 15 - protein, 8 aa, Artificial Sequence
  Description of Artificial Sequence; Note = Synthetic  Construct
  VARIANT 1, 2, 5, 6, 8: Xaa = Any Amino Acid other than Glycine
XXGAXXGX

SEQ ID NO: 16 - protein, 8 aa, Artificial Sequence
  Description of Artificial Sequence; Note = Synthetic  Construct
  VARIANT 1,2,6: Xaa = Val or Ile
  VARIANT 5,8: Xaa = any amino acid
XXGGXXGX

SEQ ID NO: 17 - protein, 8 aa, Artificial Sequence
  Description of Artificial Sequence; Note = Synthetic  Construct
  VARIANT 1,2, 6: Xaa = Val or Ile
  VARIANT 5,8: Xaa = any amino acid
XXAGXXGX

SEQ ID NO: 18 - protein, 8 aa, Artificial Sequence
  Description of Artificial Sequence; Note = Synthetic  Construct
  VARIANT 1,2,6: Xaa = Val or Ile
  VARIANT 5,8: Xaa = any amino acid
XXGAXXGX

SEQ ID NO: 19 - protein, 8 aa, Artificial Sequence
  Description of Artificial Sequence; Note = Synthetic  Construct
  VARIANT 1,2,6: Xaa = Val or Ile
XXGGLXGL

SEQ ID NO: 20 - protein, 8 aa, Artificial Sequence
  Description of Artificial Sequence; Note = Synthetic  Construct
  VARIANT 1,2,6: Xaa = Val or Ile
XXAGLXGL

SEQ ID NO: 21 - protein, 8 aa, Artificial Sequence
  Description of Artificial Sequence; Note = Synthetic  Construct
  VARIANT 1, 2, 6: Xaa = Val or Ile
XXGALXGL

SEQ ID NO: 22 - protein, 8 aa, Artificial Sequence
  Description of Artificial Sequence; Note = Synthetic  Construct
  VARIANT 1,2,5,6,8: Xaa = Any Amino Acid with a Hydrophobic Sidechain
XXGGXXGX

SEQ ID NO: 23 - protein, 8 aa, Artificial Sequence
  Description of Artificial Sequence; Note = Synthetic  Construct
  VARIANT 1,2,5,6,8: Xaa = Any Amino Acid with a Hydrophobic Sidechain
XXAGXXGX

SEQ ID NO: 24 - protein, 8 aa, Artificial Sequence
  Description of Artificial Sequence; Note = Synthetic  Construct
  VARIANT 1,2,5,6,8: Xaa = Any Amino Acid with a Hydrophobic Sidechain
XXGAXXGX

SEQ ID NO: 25 - protein, 12 aa, Artificial Sequence
  Description of Artificial Sequence; Note = Synthetic  Construct
  VARIANT 1: Xaa = is a moiety capable of optimizing interaction with the completely conserved positively charged amino acid R/K in the target
  VARIANT 2: Xaa = a flexible linker
XXVLGGVAGLLL

SEQ ID NO: 26 - protein, 847 aa, Artificial Sequence
  Description of Artificial Sequence; Note = Synthetic  Construct
MRAKGIRRNYQRLWRWGMMLLGMLMICSATEKLWVTVYYGVPVWKEAITTLFCASDAKAYDTEVHNVWATHACVPTDPNPQEVILENVTENFNMGKNNMVEQMHEDIISLWDQSLKPCVKLTPLCVTLNCTGLKKNATNTTSSNKGAMEEGEMKNCSFNVTTSIGDRMQREYALFYKLDIVPVDGDNSTRYRLISCNTSVITQACPKVSFEPIPIHYCAPAGFAILKCNNKKFNGTGPCTNVSTVQCTHGIRPVVSTQLLLNGSLAEEEVVIRSTNLSDNAKTIIVQLKDPVEIKCTRPNNNTRKSIPIGPGRAFYATGDIIGDIRQAHCNLSSTNWTNALKQIGKELRKQFKNKTIIFNQSSGGDPEIVMHSFNCGGEFFYCDSTQLFNNTWNGTEWPDDDITITLPCRIKQIINMWQEVGKAMYAPPIRGRIECSSNITGLLLTRDGGINNTNGSETFRPGGGDMRDNWRSELYKYKVVKIEPLGVAPTKAKRRVVQREKRAALGAVFLGFLGAAGSTMGAASMTLTVQARLLLSGIVQQQNNLLRAIEAQQHLLQLTVWGIKQLQARVLAVEKYLKDQQLLGIWGCSGKLICTTTVPWNASWSNKSLSEIWDNMTWMEWEREINNYTSLIYSLIEESQNQQEKNEQELLELDKWASLWNWFNITQWLWYIKIFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYRWNLLQYWSQELKNSAVNLLNATAIAVAEGTDRVIEVLQAAYRAIRHIPRRIRQGLERILL

SEQ ID NO: 27 - DNA, 2544 bp, Artificial Sequence
  Description of Artificial Sequence; Note = Synthetic  Construct
atgagagcgaaggggatcaggaggaattatcagcgcttgtggagatggggcatgatgctccttgggatgttgatgatctgtagtgctacagaaaaattgtgggtcacagtctattatggggtacctgtgtggaaagaagccatcaccactctattttgtgcatcagatgctaaagcatatgatacagaggtacataatgtttgggccacacatgcctgtgtacccacagaccccaacccacaagaagtaatattggaaaatgtgacagaaaattttaacatggggaaaaataacatggtagaacagatgcatgaggatataatcagtttatgggatcaaagcctaaagccatgcgtaaaattaaccccactctgtgttactttaaattgcactggtctgaagaagaatgctactaataccactagtagtaacaagggagcgatggaggaaggagaaatgaaaaactgctctttcaatgtcaccacaagcataggagataggatgcagagagaatatgcacttttttataaacttgatatagtaccagtagatggtgataatagtaccagatataggttgataagttgcaacacctcagtcattacacaggcttgtccaaaggtatcctttgagccaattcccatacattattgtgccccggctggttttgcgattctaaagtgtaacaataagaagttcaatggaacaggaccatgtacaaatgtcagcacagtacaatgtacacatggaattaggccagtagtatcgactcaactgctgttaaatggcagtctagcagaagaagaggtagtaattagatctaccaatctctcggacaatgctaaaaccataatagtacagctaaaagaccctgtagaaattaagtgtacaagacccaacaacaatacaagaaaaagtatacctataggaccagggagagcattttatgcaacaggagacataataggagatataagacaagcacattgtaaccttagttcaacaaactggactaacgctttaaaacagataggtaaagaattaagaaaacagtttaagaataaaacaataatctttaatcaatcctcaggaggggacccagaaattgtaatgcacagctttaattgtggaggggaatttttctactgtgattcaacacaactgtttaataatacttggaatggtactgaatggccagatgacgatataactatcacactcccatgcagaataaaacaaattataaacatgtggcaggaagtaggaaaagcaatgtatgcccctcccatcagaggacgaattgaatgttcatcaaatattacaggactactactaacaagagatggtggtattaataacacgaatgggagcgagaccttcagacctggaggaggagatatgagggacaattggagaagtgaattatataaatataaagtagtaaaaatagaaccattaggagtagcacccaccaaggcaaagagaagagtggtgcagagagaaaaaagagcagcattaggagctgtgttccttgggttcttaggagcagcaggaagcactatgggcgcagcgtcgatgacgctgacggtacaggccagactattgttgtctggtatagtgcaacagcagaacaatttgctgagggctattgaggcgcaacagcatctgttgcaactcacagtctggggcatcaagcagctccaggcaagagtcctggctgtggaaaaatacctaaaggatcaacagctcctggggatttggggttgctctggaaaactcatttgcaccactactgtgccctggaatgctagttggagtaataaatctctgagtgagatttgggataacatgacctggatggagtgggaaagagaaattaacaattacacaagcttaatatacagcttaattgaagaatcgcaaaaccaacaagagaagaatgaacaagaattattagaattggataaatgggcaagtctgtggaattggtttaacataacacaatggctgtggtatataaaaatattcataatgatagtaggaggcttggtaggtttaagaatagtttttgctgtactctctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatatcggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataa

SEQ ID NO: 28 - protein, 11 aa, Artificial Sequence
  Description of Artificial Sequence; Note = Synthetic  Construct
  VARIANT 4: Xaa = Val or Leu
  VARIANT 7: Xaa = Val or Ala
EGGXGGXGLLL

SEQ ID NO: 29 - protein, 29 aa, Artificial Sequence
  Description of Artificial Sequence; Note = Synthetic  Construct
TNWLWYIKLFIMIVGGLVGLRIVFAKKKC

SEQ ID NO: 30 - protein, 26 aa, Artificial Sequence
  Description of Artificial Sequence; Note = Synthetic  Construct
QPMALIVGGLVGLLLFIGLGIFFCVR

SEQ ID NO: 31 - protein, 8 aa, Artificial Sequence
  Description of Artificial Sequence; Note = Synthetic  Construct
HIGFGGIF

SEQ ID NO: 32 - protein, 8 aa, Artificial Sequence
  Description of Artificial Sequence; Note = Synthetic  Construct
VGGLLGNC

SEQ ID NO: 33 - protein, 10 aa, Artificial Sequence
  Description of Artificial Sequence; Note = Synthetic  Construct
IVGGLVGLLL

SEQ ID NO: 34 - protein, 15 aa, Artificial Sequence
  Description of Artificial Sequence; Note = Synthetic  Construct
  VARIANT 14: Xaa = a flexible glycyl linker of any length such as 1, 2, 3, 4, 5, 6, 7, 8, or 9
  VARIANT 15: Xaa = Arginines, of any length, such as 1, 2, 3, 4, 5, 6, 7, 8, or 9
EGGIVGGVAGLLLXX

SEQ ID NO: 35 - protein, 13 aa, Artificial Sequence
  Description of Artificial Sequence; Note = Synthetic  Construct
FMIVGGLVGLRIV

SEQ ID NO: 36 - protein, 13 aa, Artificial Sequence
  Description of Artificial Sequence; Note = Synthetic  Construct
ALVLGGVAGLLLF